(19) United States
(12) Patent Application Publication
(10) Pub. No.: US 2025/0073217 A1
(43) Pub. Date: Mar. 6, 2025

(54) SMALL MOLECULE ADRENORECEPTOR ANTAGONISTS AND USES THEREOF

(71) Applicant: ARIZONA BOARD OF REGENTS ON BEHALF OF THE UNIVERSITY OF ARIZONA, Tucson, AZ (US)

(72) Inventors: Iman Daryaei, Tucson, AZ (US); John Streicher, Tucson, AZ (US); Abigail Schwarz, Tucson, AZ (US)

(21) Appl. No.: 18/725,851

(22) PCT Filed: Jan. 4, 2023
(86) PCT No.: PCT/US2023/010107

§ 371 Date: Jul. 1, 2024

Related U.S. Application Data
(60) Provisional application No. 63/296,248, filed on Jan. 4, 2022.

(51) Int. Cl. A61K 31/4468; A61K 31/4525; A61K 31/4535; A61K 31/485

(52) U.S. Cl. CPC A61K 31/4468; A61K 31/4525; A61K 31/4535; A61K 31/485

(57) ABSTRACT

This invention is in the field of medicinal chemistry. In particular, the invention relates to a new class of small-molecules having a piperdinyl-formamide (or similar) structure which function as adrenoreceptor antagonists, and their use as therapeutics for the treatment and/or prevention of pain and related conditions. In addition, the present invention provides compositions comprising a mixture of opioid receptor agonist compounds and such adrenoreceptor antagonists for the treatment and/or prevention of pain and related conditions.

### CROSS-REFERENCE TO RELATED APPLICATION

The present application claims priority to U.S. Provisional Patent Application No. 63/296,248, filed Jan. 4, 2022, which is hereby incorporated by reference in its entirety.

### FIELD OF THE INVENTION

This invention is in the field of medicinal chemistry. In particular, the invention relates to a new class of small-molecules having a piperdinyl-formamide (or similar) structure which function as adrenoreceptor antagonists, and their use as therapeutics for the treatment and/or prevention of pain and related conditions. In addition, the present invention provides compositions comprising a mixture of opioid receptor agonist compounds and such adrenoreceptor antagonists for the treatment and/or prevention of pain and related conditions.

### INTRODUCTION

Pain is caused by a highly complex perception of an aversive or unpleasant sensation, and the management of pain is a major challenge as millions of people all over the world suffer from pain every day. There are two major categories of pain, namely, nociceptive pain and neuropathic pain. Each of these types of pain can be either acute or chronic.

Opioids continue to be the backbone for the treatment of these pain states. However, constant opioid treatment is accompanied with serious undesirable effects including drowsiness and mental clouding, nausea and emesis, constipation and in many cases dependence and addiction. Continuous use of opioid therapy also develops analgesic tolerance and hyperalgesia in many patients. These unwanted effects significantly diminish the patients' quality of life. The mechanisms for these side effects are still largely unclear. However, one particular side effect, namely addiction, is a cause for great concern in pain treatment using opioids.

Prescription opioids can be highly addictive and are widely misused. It has been estimated that the total economic cost of opioid misuse in the United States is $78.5 billion per year. The opioid crisis has been exacerbated by over-prescription of opioid pain-relievers for the treatment of pain. Due at least in part due to over-prescription and additive nature of opioids, some who experience chronic pain may suffer undertreatment. In fact, in many parts of the developing world, access to opioids even for acute pain and/or cancer pain can be restricted due to concerns over addiction and overdose. Even in the United States, some patients can suffer from an undertreatment of pain. For example, patients with cognitive impairment and the elderly can be especially susceptible to the central nervous system effects of traditional opioids such as morphine and in some cases are not prescribed enough to meet their pain management needs.

Accordingly, there is a need for safe and effective analgesic compositions for the treatment of pain that do not suffer from the side effects of traditional opioids.

The present invention addresses these needs.

### SUMMARY OF THE INVENTION

Experiments conducted during the course of developing embodiments determined that a new class of small-molecules having a piperdinyl-formamide (or similar) structure which function as adrenoreceptor antagonists. Experiments further determined that a composition that includes a mixture of an opioid receptor agonist compound in combination with such adrenoreceptor antagonist small-molecules provide benefits of opioid receptor agonist properties with a significant reduction or elimination of additive side effects of opioid receptor agonist compound.

Accordingly, the present invention relates to a new class of small-molecules having a piperdinyl-formamide (or similar) structure which function as adrenoreceptor antagonists, and their use as therapeutics for the treatment and/or prevention of pain and related conditions. In addition, the present invention provides compositions comprising a mixture of opioid receptor agonist compounds and such adrenoreceptor antagonists for the treatment and/or prevention of pain and related conditions.

Certain piperdinyl-formamide (or similar) compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, adrenoreceptor modulator compounds encompassed within Formula II are provided:

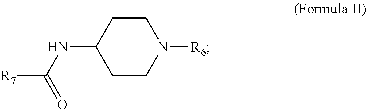

including pharmaceutically acceptable salts, solvates, and/or prodrugs thereof.

Formula II is not limited to a particular chemical moiety for R6 and R7. In some embodiments, the particular chemical moiety for R6 and R7 independently include any chemical moiety that permits the resulting compound to inhibit and/or antagonize adrenoeceptor activity. In some embodiments, the particular chemical moiety for R6 and R7 independently include any chemical moiety that permits the resulting compound to inhibit and/or antagonize α2A adrenoeceptor activity. In some embodiments, the particular chemical moiety for R6 and R7 independently include any chemical moiety that permits the resulting compound to inhibit and/or antagonize opioid receptor activity. In some embodiments, the particular chemical moiety for R6 and R7 independently include any chemical moiety that permits the resulting compound to inhibit and/or antagonize α2A adrenoreceptor activity. In some embodiments, the particular chemical moiety for R6 and R7 independently include any chemical moiety that permits the resulting compound to prevent and/or attenuate the addictive properties of opioid receptor agonist compounds. In some embodiments, the particular chemical moiety for R6 and R7 independently include any chemical moiety that permits the resulting compound to treat, prevent or attenuate pain. In some embodiments, the particular chemical moiety for R6 and R7 independently include any chemical moiety that permits the resulting compound to treat, prevent or attenuate nociceptive pain. In some embodiments, the particular chemical moiety for R6 and R7 independently include any chemical moiety that permits the resulting compound to treat, prevent or attenuate neuropathic pain. In some embodiments, the particular chemical moiety for R6 and R7 independently include any chemical moiety that permits the resulting compound to provide non-addictive pain relief to a subject upon co-administration with an opioid receptor agonist compound. In some embodiments, the particular chemical moiety for R6 and R7 independently include any chemical moiety that permits the resulting compound to provide direct treatment for opioid addiction disorder to a subject upon co-administration with an opioid receptor agonist compound.

In some embodiments, the adrenoreceptor modulator is an adrenoreceptor antagonist. In some embodiments, the adrenoreceptor modulator is an α2A adrenoreceptor antagonist.

In some embodiments, R6 is selected from

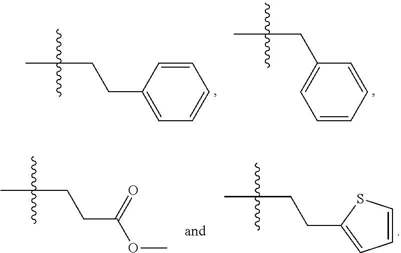

In some embodiments,

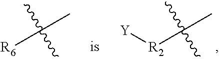

R2 is C₁₋₆ alkylene; Y is optionally substituted aryl, optionally substituted heteroaryl, or a moiety of the formula —C(═O)—X¹; X¹ is —OR₃ or —NR⁴R5; and each of R³, R⁴ and R⁵ is independently H or C₁₋₁₀ alkyl.

In some embodiments, R2 is ethylene.

In some embodiments, Y is selected from phenyl, thiophen-2-yl, and a moiety of the formula —C(═O)—OR₃, where R³ is C₁₋₁₀ alkyl.

In some embodiments, R7 is selected from

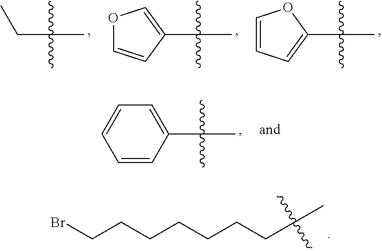

In some embodiments, R7 is selected from C₁₋₁₀ alkyl, C₁₋₁₀ haloalkyl, optionally substituted aryl, and optionally substituted heteroaryl.

In some embodiments, R7 is selected from ethyl, 7-bromoheptyl, fur-2-yl, fur-3-yl, and phenyl.

In some embodiments, the compound is selected from:

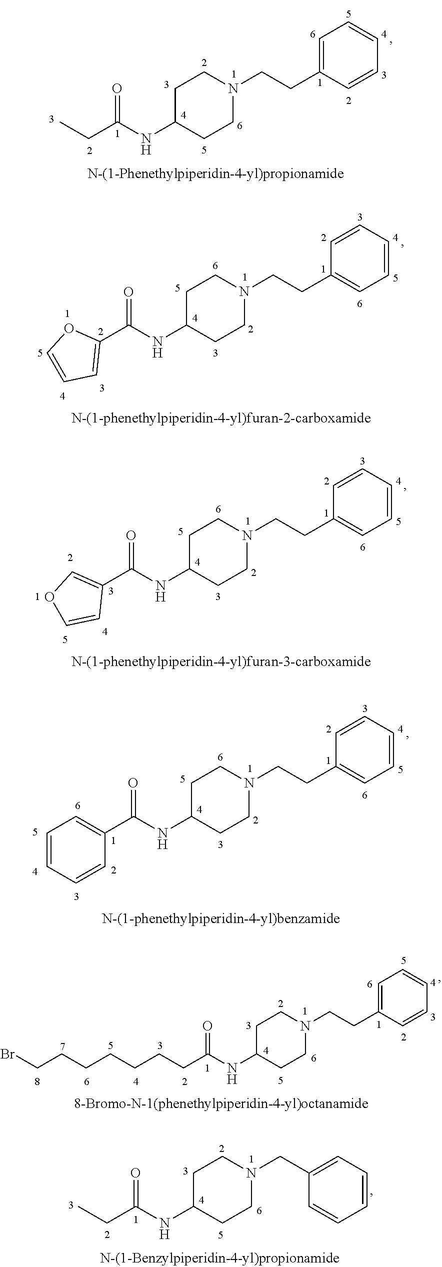 [SMILES: CCC(=O)NC1CCN(CCc2ccccc2)CC1 ; O=C(NC1CCN(CCc2ccccc2)CC1)c1ccco1 ; O=C(NC1CCN(CCc2ccccc2)CC1)c1ccoc1 ; O=C(NC1CCN(CCc2ccccc2)CC1)c1ccccc1 ; O=C(CCCCCCCBr)NC1CCN(CCc2ccccc2)CC1 ; CCC(=O)NC1CCN(Cc2ccccc2)CC1]

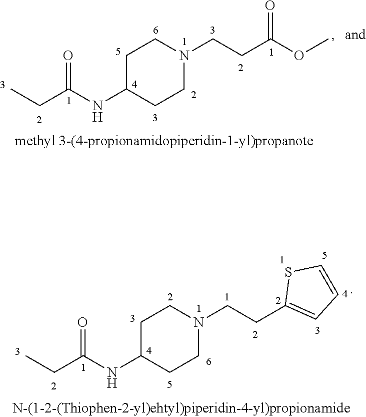

The invention further provides processes for preparing any of the compounds of the present invention.

The piperdinyl-formamide (or similar) compounds described herein can be considered as potential therapeutics for the treatment, prevention, and/or amelioration of pain (e.g., nociceptive pain; neuropathic pain).

The piperdinyl-formamide (or similar) compounds described herein can be considered as potential therapeutics for the treatment, prevention, and/or amelioration of pain (e.g., nociceptive pain; neuropathic pain) when administered in combination with opioid receptor agonist compounds (e.g., morphine, fentanyl, carfentanyl, pentazocine, butorphanol, nalbuphine, buprenorphine, sufentanil, alfentanil, tramadol, remifentanil, hydrocodone, oxycodone, hydromorphone, oxymorphone, or similar clinically used opioid receptor agonist drug).

The piperdinyl-formamide (or similar) compounds described herein can be considered as potential therapeutics for the treatment, prevention, and/or amelioration of addiction related to opioid receptor agonist compounds.

The piperdinyl-formamide (or similar) compounds described herein can be considered as potential therapeutics for the treatment, prevention, and/or amelioration of conditions associated with adrenoreceptor activity.

The piperdinyl-formamide (or similar) compounds described herein can be considered as potential therapeutics for the treatment, prevention, and/or amelioration of conditions associated with α2A adrenoreceptor activity.

Some aspects of the invention provide a composition that includes a mixture of an opioid receptor agonist compound in combination with an adrenoreceptor modulator compound. Surprisingly and unexpectedly, the present inventors have found that such a combination provides benefits of opioid receptor agonist properties with a significant reduction or elimination of additive side effects of opioid receptor agonist compound.

In some embodiments, said opioid receptor agonist compound comprises morphine, fentanyl, carfentanyl, pentazocine, butorphanol, nalbuphine, buprenorphine, sufentanil, alfentanil, tramadol, remifentanil, hydrocodone, oxycodone, hydromorphone, oxymorphone, or similar clinically used opioid receptor agonist drug.

Still in other embodiments, said adrenoreceptor modulator compound is an α2A adrenoreceptor antagonist.

Yet in one particular embodiment, said adrenoreceptor modulator compound is of the formula:

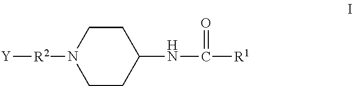

where
R¹ is C₁₋₁₀ alkyl, C₁₋₁₀ haloalkyl, optionally substituted aryl, or optionally substituted heteroaryl;
R² is C₁₋₆ alkylene; and
Y is optionally substituted aryl, optionally substituted heteroaryl, or a moiety of the formula —C(═O)—X¹, wherein X¹ is —OR₃ or —NR⁴R⁵, where each of R³, R⁴ and R⁵ is H or C₁₋₁₀ alkyl.

In some embodiments, the adrenoreceptor modulator is an adrenoreceptor antagonist. In some embodiments, the adrenoreceptor modulator is an α2A adrenoreceptor antagonist.

In some embodiments, R¹ is selected from the group consisting of ethyl, 7-bromoheptyl, fur-2-yl, fur-3-yl, and phenyl. Still in other embodiments, R² is ethylene. In further embodiments, Y is selected from the group consisting of phenyl, thiophen-2-yl, and a moiety of the formula —C(═O)—OR₃, where R³ is C₁₋₁₀ alkyl.

In other embodiments, said adrenoreceptor modulator compound comprises N-(1-phenethylpiperidin-4-yl)propionamide (Compound 1), N-(1-phenethylpiperidin-4-yl)furan-2-carboxamide (Compound 2), N-(1-phenethylpiperidin-4-yl)furan-3-carboxamide (Compound 3), N-(1-(2-(thiophen-2-yl)ethyl) piperidin-4-yl)propionamide (Compound 4), or a mixture thereof.

Another aspect of the invention provides a method for treating pain or addiction disorder in a subject. The method includes administering to a subject in need of such a treatment a therapeutically effective amount of a composition comprising an opioid receptor agonist compound and an adrenoreceptor modulator compound. Such a composition provides non-addictive pain relief, and/or direct treatment for opioid addiction disorder.

In some embodiments, the opioid receptor agonist compound comprises morphine, fentanyl, carfentanyl, pentazocine butorphanol, nalbuphine, buprenorphine, sufentanil, alfentanil, tramadol, remifentanil, hydrocodone, oxycodone, hydromorphone, oxymorphone, or similar clinically used opioid receptor agonist drug.

Yet in other embodiments, said adrenoreceptor modulator compound is an α2A adrenergic antagonist. In other embodiments, said adrenoreceptor modulator compound is an opioid receptor modulator.

Still in other embodiments, said adrenoreceptor modulator compound is of the formula:

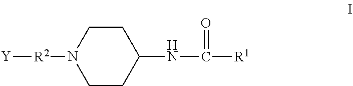

where R¹ is C₁₋₁₀ alkyl, C₁₋₁₀ haloalkyl, optionally substituted aryl, or optionally substituted heteroaryl; R² is C₁₋₆ alkylene; and Y is optionally substituted aryl, optionally substituted heteroaryl, or a moiety of the formula —C(═O)—X¹, wherein X¹ is —OR₃ or —NR⁴R⁵, where each of R³, R⁴ and R⁵ is H or C₁₋₁₀ alkyl. In some embodiments, R¹ is selected from the group consisting of ethyl, 7-bromoheptyl, fur-2-yl, fur-3-yl, and phenyl. Still in other embodiments, R² is ethylene. In further embodiments, Y is selected from the group consisting of phenyl, thiophen-2-yl, and a moiety of the formula —C(═O)—OR₃, where R³ is C₁₋₁₀ alkyl.

In some embodiments, the adrenoreceptor modulator is an adrenoreceptor antagonist. In some embodiments, the adrenoreceptor modulator is an α2A adrenoreceptor antagonist.

In further embodiments, said adrenoreceptor modulator compound comprises N-(1-phenethylpiperidin-4-yl)propionamide (Compound 1), N-(1-phenethylpiperidin-4-yl)furan-2-carboxamide (Compound 2), N-(1-phenethylpiperidin-4-yl)furan-3-carboxamide (Compound 3), N-(1-(2-(thiophen-2-yl)ethyl) piperidin-4-yl)propionamide (Compound 4), or a mixture thereof.

Still in other embodiments, said pain is chronic pain. In other embodiments, said pain is acute pain. Yet in other embodiments, said pain is nociceptive pain. In further embodiments, said pain is neuropathic pain.

Yet another aspect of the invention provides a composition comprising an opioid receptor agonist compound and a second therapeutically active compound, wherein said second therapeutically active compound has an adrenoreceptor modulator activity and an opioid receptor modulating activity.

In some embodiments, said second biologically active compound is of the formula:

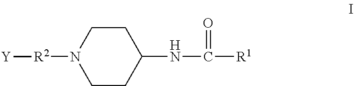

where R¹ is C₁₋₁₀ alkyl, C₁₋₁₀ haloalkyl, optionally substituted aryl, or optionally substituted heteroaryl; R² is C₁₋₆ alkylene; and Y is optionally substituted aryl, optionally substituted heteroaryl, or a moiety of the formula —C(═O)—X¹, wherein X¹ is —OR₃ or —NR⁴R⁵, where each of R³, R⁴ and R⁵ is H or C₁₋₁₀ alkyl. In some instances, R¹ is selected from the group consisting of ethyl, 7-bromoheptyl, fur-2-yl, fur-3-yl, and phenyl. Still in other instances, R² is ethylene. In further instances, Y is selected from the group consisting of phenyl, thiophen-2-yl, and a moiety of the formula —C(═O)—OR₃, where R³ is C₁₋₁₀ alkyl. Still in other embodiments, said second biologically active compound comprises N-(1-phenethylpiperidin-4-yl)propionamide, N-(1-phenethylpiperidin-4-yl)furan-2-carboxamide, N-(1-phenethylpiperidin-4-yl)furan-3-carboxamide, or N-(1-(2-(thiophen-2-yl)ethyl) piperidin-4-yl)propionamide.

The invention also provides pharmaceutical compositions comprising the compounds of the invention in a pharmaceutically acceptable carrier.

The invention also provides kits comprising a compound of the invention and instructions for administering the compound to an animal. The kits may optionally contain other therapeutic agents, e.g., opioid receptor agonist compounds (e.g., morphine, fentanyl, carfentanyl, pentazocine, butorphanol, nalbuphine, buprenorphine, sufentanil, alfentanil, tramadol, remifentanil, hydrocodone, oxycodone, hydromorphone, oxymorphone, or similar clinically used opioid receptor agonist drug).

The present disclosure further provides bifunctional compounds that function to recruit endogenous proteins to an E3 Ubiquitin Ligase for degradation, and methods of using the same. In particular, the present disclosure provides bifunctional or proteolysis targeting chimeric (PROTAC) compounds, which find utility as modulators of targeted ubiquitination of a variety of polypeptides and other proteins, which are then degraded and/or otherwise inhibited. An exemplary advantage of the compounds provided herein is that a broad range of pharmacological activities is possible, consistent with the degradation/inhibition of targeted polypeptides from virtually any protein class or family. In addition, the description provides methods of using an effective amount of the compounds as described herein for the treatment, prevention and/or amelioration of pain (e.g., nociceptive pain; neuropathic pain).

In an additional aspect, the disclosure provides bifunctional or PROTAC compounds, which comprise an E3 Ubiquitin Ligase binding moiety (e.g., a ligand for an E3 Ubquitin Ligase or “ULM” group), and a moiety that binds a target protein (e.g., a protein/polypeptide targeting ligand or “PTM” group) (e.g., adrenoreceptor) such that the target protein/polypeptide is placed in proximity to the ubiquitin ligase to effect degradation (and inhibition) of that protein (e.g., inhibit adrenoreceptor activity). In certain embodiments, the PTM is any of the compounds as described herein showing inhibitory activity against adrenoreceptor activity. In certain embodiments, the PTM is any of the compounds as described herein showing inhibitory activity against adrenoreceptor activity. In some embodiments, the ULM is a VHL, cereblon, mouse double minute 2 (MDM2), and/or inhibitor of apoptosis protein (IAP) E3 ligase binding moiety. For example, the structure of the bifunctional compound can be depicted as PTM-ULM.

The respective positions of the PTM and ULM moieties, as well as their number as illustrated herein, is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as PTM-L-ULM, where PTM is a protein/polypeptide targeting moiety (e.g., any of the compounds as described herein showing inhibitory activity against pain) (e.g., any of the compounds as described herein showing inhibitory activity specifically against adrenoreceptor activity), L is a linker, and ULM is a VHL, cereblon, MDM2, or IAP E3 ligase binding moiety binding moiety.

Such embodiments are not limited to a specific type of linker. In some embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker may be asymmetric or symmetrical. In some embodiments, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

The ULM group and PTM group may be covalently linked to the linker group through any group which is appropriate and stable to the chemistry of the linker. In exemplary aspects of the present invention, the linker is independently covalently bonded to the ULM group and the PTM group in certain embodiments through an amide, ester, thioester, keto group, carbamate (urethane), carbon or ether, each of which groups may be inserted anywhere on the ULM group and PTM group to provide maximum binding of the ULM group on the ubiquitin ligase and the PTM group on the target protein to be degraded. In certain aspects where the PTM group is a ULM group, the target protein for degradation may be the ubiquitin ligase itself. In certain exemplary aspects, the linker may be linked to an optionally substituted alkyl, alkylene, alkene or alkyne group, an aryl group or a heterocyclic group on the ULM and/or PTM groups.

In certain embodiments, the compounds as described herein comprise multiple ULMs, multiple PTMs, multiple chemical linkers, or any combinations thereof.

In some embodiments, the present invention provides a method of ubiquitinating/degrading adrenoreceptor in a cell comprising administering a bifunctional compound as described herein comprising an ULM and a PTM, in certain embodiments linked through a linker moiety, as otherwise described herein, wherein the ULM is coupled to the PTM and wherein the ULM recognizes a ubiquitin pathway protein and the PTM recognizes the target protein such that degradation of the target protein occurs when the target protein is placed in proximity to the ubiquitin ligase, thus resulting in degradation/inhibition of the effects of the target protein and the control of protein levels. The control of protein levels afforded by the present invention provides treatment of a disease state or condition, which is modulated through the target protein by lowering the level of that protein in the cells of a patient.

### BRIEF DESCRIPTION OF THE DRAWINGS

FIG. 1 shows results of mice CPP chamber test.

FIGS. 2A-2C show results of female mice tail flick test, male mice tail flick test, and combined mice tail flick test, respectively.

### DEFINITIONS

As used herein, the terms “treat”, “treating”, or “treatment” refer to both therapeutic treatment and prophylactic or preventative measures, with the objective of preventing, reducing, slowing down (lessen), inhibiting, or eliminating an undesired physiological change, symptom, or disorder, such as the development or spread of pain. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. For example, treatment with the composition comprising the calcium channel modulator of the invention may include reduction of pain. “Treatment” can also mean prolonging survival as compared to expected survival if not receiving treatment. Those in need of treatment include those already with a condition or disorder as well as those prone to have a condition or disorder or those in which a condition or disorder is to be prevented or onset delayed. Optionally, the subject or patient may be identified (e.g., diagnosed) as one suffering from the disease or condition (e.g., pain) prior to administration of the modulator of the invention.

A “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disease or condition being treated and the severity of the disease or condition; the specific composition employed; the age, body weight, general health, sex and diet of the patient; the time of administration; the route of administration: the rate of excretion of the specific compound employed; the duration of the treatment; drugs used in combination or coincidental with the specific modulator employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adults and new born subjects, as well as fetuses, whether male or female, are intended to be included. A “patient” is a subject afflicted with a disease, disorder, or condition, (e.g., pain). The term “patient” includes human and veterinary subjects.

The terms “administering” and “administration” refer to methods of providing a pharmaceutical composition to a subject. Such methods are well known to those skilled in the art. Pharmaceutical compositions can be administered in a number of ways depending on whether local or systemic treatment is desired, and on the area to be treated. Administration include, but is not limited to, administering the compositions topically (including ophthalmically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, intrathecally, extracorporeally, transdermally, or the like.

As described above, the compositions and/or compounds can be administered to a subject in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient (e.g., the calcium channel modulator) and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution may be about 5 to about 8, such as from about 7 to about 7.5. Further carriers include sustained release preparations such as semi-permeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more desirable depending upon, for instance, the route of administration and concentration of composition being administered. Other compounds can be administered according to standard procedures used by those skilled in the art.

Pharmaceutical compositions can include additional carriers, as well as thickeners, diluents, buffers, preservatives, surface active agents and the like in addition to the compounds disclosed herein. Pharmaceutical those can also include one or more additional active ingredients such as antimicrobial agents, anti-inflammatory agents, anesthetics, and the like.

For example, pharmaceutical compositions for parenteral administration include sterile aqueous or non-aqueous solutions, suspensions, and emulsions. Examples of non-aqueous solvents are propylene glycol, polyethylene glycol, vegetable oils such as olive oil, fish oils, and injectable organic-esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions for topical administration include but are not limited to, ointments, lotions, creams, gels, drops, suppositories, sprays, liquids and powders. Conventional pharmaceutical carriers, aqueous, powder or oily bases, thickeners and the like may be necessary or desirable. Pharmaceutical compositions for oral administration include, but are not limited to, powders or granules, suspensions or solutions in water or non-aqueous media, capsules, sachets, or tablets. Thickeners, flavorings, diluents, emulsifiers, dispersing aids or binders may be desirable.

“Alkyl” refers to a saturated linear monovalent hydrocarbon moiety or a saturated branched monovalent hydrocarbon moiety. Exemplary alkyl group include, but are not limited to, methyl, ethyl, n-propyl, 2-propyl, tert-butyl, pentyl, and the like.

“Alkylene” refers to a saturated linear saturated divalent hydrocarbon moiety or a branched saturated divalent hydrocarbon moiety. Exemplary alkylene groups include, but are not limited to, methylene, ethylene, propylene, butylene, iso-butylene, pentylene, and the like.

“Aryl” refers to a monovalent mono-, bi- or tricyclic aromatic hydrocarbon moiety of 6 to 15 ring atoms which is optionally substituted with one or more substituents. More specifically the term aryl includes, but is not limited to, phenyl, 1-naphthyl, and 2-naphthyl, and the derivatives thereof. When substituted, the aryl group typically contains one, two or three substituents within the ring structure. Moreover, when two or more substituents are present in an aryl group, each substituent is independently selected. Exemplary substituents for the aryl group include, but are not limited to, deuterium, alkyl, haloalkyl, heteroalkyl, halo, nitro, cyano, optionally substituted phenyl, heteroaryl, haloalkoxy, —OR′ (where R′ is H, alkyl or a phenol protecting group) and carboxyl (i.e., a moiety of the formula —COX, where X is —OR^{a} or —NR^{b}R^{c}, where each of R^{a}, R^{b}, R^{c} is independently H, alkyl, or a corresponding protecting group.

“Aralkyl” refers to a moiety of the formula —R^{b}R^{c} where R^{b} is an alkylene group and R^{c} is an optionally substituted aryl group as defined herein. Exemplary aralkyl groups include, but are not limited to, benzyl, phenylethyl, (halo-substituted phenyl)ethyl, and the like.

The terms “halo,” “halogen” and “halide” are used interchangeably herein and refer to fluoro, chloro, bromo, or iodo.

“Haloalkyl” refers to an alkyl group as defined herein in which one or more hydrogen atom is replaced by same or different halo atoms. The term “haloalkyl” also includes perhalogenated alkyl groups in which all alkyl hydrogen atoms are replaced by halogen atoms. Exemplary haloalkyl groups include, but are not limited to, —CH₂Cl, —CF₃, —CH₂CF₃, —CH₂CCl₃, and the like.

The term “heteroaryl” means a monovalent monocyclic or bicyclic aromatic moiety of 5 to 12 ring atoms containing one, two, or three ring heteroatoms selected from N, O, or S, the remaining ring atoms being C. Exemplary heteroaryl includes, but is not limited to, pyridyl, furanyl, thiophenyl, thiazolyl, isothiazolyl, triazolyl, imidazolyl, isoxazolyl, pyrrolyl, pyrazolyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, and benzodiazepin-2-one-5-yl, and the like. The heteroaryl ring can optionally be substituted with one or more substituents, typically one or two substituents. When two or more substituents are present in heteroaryl, each substituent is independently selected. Exemplary substituents for heteroaryl include, but are not limited to, substituents described for aryl group above.

As used herein, the term “opioid receptor agonist compound” refers to any compounds that have opioid receptor agonist activity. In some embodiments, the opioid receptor agonist compounds have analgesic effect. Still in other embodiments, the opioid receptor agonist compound has μ-opioid receptor agonist activity. Exemplary opioid receptor agonist compounds include, but are not limited to, naturally occurring opioid receptor agonist compounds and synthetic opioid receptor agonist compounds such as morphine, fentanyl, carfentanyl, pentazocine, butorphanol, nalbuphine, buprenorphine, sufentanil, alfentanil, tramadol, remifentanil, hydrocodone, oxycodone, hydromorphone, oxymorphone, pethidine, levorphanol, methadone, dextropropoxyphene, as well as other similar clinically used opioid receptor agonist drugs. However, it should be appreciated that any opioid receptor agonist compound having μ-opioid receptor agonist activity disclosed above can be used in compositions and methods of the invention. μ-Opioid receptor agonist activity can be readily determined for any compound using methods and procedures that are well known to one skilled in the art.

### DETAILED DESCRIPTION OF THE INVENTION

Experiments conducted during the course of developing embodiments determined that a new class of small-molecules having a piperdinyl-formamide (or similar) structure which function as adrenoreceptor antagonists. Experiments further determined that a composition that includes a mixture of an opioid receptor agonist compound in combination with such adrenoreceptor antagonist small-molecules provide benefits of opioid receptor agonist properties with a significant reduction or elimination of additive side effects of opioid receptor agonist compound.

Accordingly, the present invention relates to a new class of small-molecules having a piperdinyl-formamide (or similar) structure which function as adrenoreceptor antagonists, and their use as therapeutics for the treatment and/or prevention of pain and related conditions. In addition, the present invention provides compositions comprising a mixture of opioid receptor agonist compounds and such adrenoreceptor antagonists for the treatment and/or prevention of pain and related conditions.

Use of opioid receptor agonist compounds is a method of choice in treating a wide variety of pain. Unfortunately, many commonly used opioid receptor agonist compounds for pain treatment are also highly addictive due in part to their activation of μ-opioid receptor. For example, a μ-opioid receptor agonist morphine has higher antinociceptive activity but is also highly addictive. Moreover, continuous use of opioid receptor agonist compounds also leads to analgesic tolerance and hyperalgesia in many patients.

Some aspects of the invention provide a composition that is useful in treating pain with a significantly reduced or eliminated addiction potential. In some embodiments, the compositions of the invention have at least about 10% or more, typically at least about 20% or more, often at least about 25% or more, and most often at least about 50% or more reduction in addictive property. One example of measuring addictive property is disclosed in the Examples section where the amount of time mice spend in conditioned place preference (CPP) chambers after administration of an opioid receptor agonist compound only vs, a composition of the invention. In this manner, reduction in addictive property can be determined by reduction in the amount of time rat spends in CPP chambers as illustrated in the Examples section.

One particular aspect of the invention provides a composition comprising an opioid receptor agonist compound and an adrenoreceptor antagonist compound. In general, any opioid receptor agonist compound that has an analgesic effect and having an agonist activity as disclosed herein can be used in the composition of the invention. In some embodiments, the opioid receptor agonist compound is any opioid receptor agonist compound known to one skilled in the art having analgesic activity. Still in another embodiment, the opioid receptor agonist compound is any drug, or a combination of two or more drugs, listed in the orange book of U.S. Food and Drug Administration (“FDA”) and having an opioid receptor agonist activity. Exemplary opioid receptor agonist compounds include, but are not limited to, morphine, fentanyl, carfentanyl, pentazocine, butorphanol, nalbuphine, buprenorphine, sufentanil, alfentanil, tramadol, remifentanil, hydrocodone, oxycodone, hydromorphone, oxymorphone, pethidine, levorphanol, methadone, dextropropoxyphene, as well as other similar clinically used opioid receptor agonist drugs.

Opioid receptor agonist compounds work in the brain at specific opiate receptors. Several types of the opiate receptors are known, but the main receptor for pain is the μ-opioid receptor or simply μ-receptor. Accordingly, in some embodiments, the opioid receptor agonist compound is a μ-receptor agonist.

Some aspects of the invention provide a composition that includes a mixture of an opioid receptor agonist compound in combination with an adrenoreceptor modulator compound. Surprisingly and unexpectedly, the present inventors have found that such a combination provides benefits of opioid receptor agonist properties with a significant reduction or elimination of additive side effects of opioid receptor agonist compound.

In some embodiments, said opioid receptor agonist compound comprises morphine, fentanyl, carfentanyl, pentazocine, butorphanol, nalbuphine, buprenorphine, sufentanil, alfentanil, tramadol, remifentanil, hydrocodone, oxycodone, hydromorphone, oxymorphone, or similar clinically used opioid receptor agonist drug.

Still in other embodiments, said adrenoreceptor modulator compound is an α2A adrenoreceptor antagonist.

Yet in one particular embodiment, said adrenoreceptor modulator compound is of the formula:

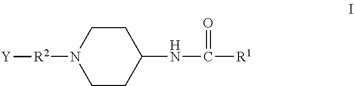

where
R¹ is C₁₋₁₀ alkyl, C₁₋₁₀ haloalkyl, optionally substituted aryl, or optionally substituted heteroaryl;
R² is C₁₋₆ alkylene; and
Y is optionally substituted aryl, optionally substituted heteroaryl, or a moiety of the formula —C(═O)—X¹, wherein X¹ is —OR₃ or —NR⁴R⁵, where each of R³, R⁴ and R⁵ is H or C₁₋₁₀ alkyl.

In some embodiments, R¹ is selected from the group consisting of ethyl, 7-bromoheptyl, fur-2-yl, fur-3-yl, and phenyl. Still in other embodiments, R² is ethylene. In further embodiments, Y is selected from the group consisting of phenyl, thiophen-2-yl, and a moiety of the formula —C(═O)—OR₃, where R³ is C₁₋₁₀ alkyl.

In other embodiments, said adrenoreceptor modulator compound comprises N-(1-phenethylpiperidin-4-yl)propionamide (Compound 1), N-(1-phenethylpiperidin-4-yl)furan-2-carboxamide (Compound 2), N-(1-phenethylpiperidin-4-yl)furan-3-carboxamide (Compound 3), N-(1-(2-(thiophen-2-yl)ethyl) piperidin-4-yl)propionamide (Compound 4), or a mixture thereof.

Another aspect of the invention provides a method for treating pain or addiction disorder in a subject. The method includes administering to a subject in need of such a treatment a therapeutically effective amount of a composition comprising an opioid receptor agonist compound and an adrenoreceptor modulator compound. Such a composition provides non-addictive pain relief, and/or direct treatment for opioid addiction disorder.

In some embodiments, the opioid receptor agonist compound comprises morphine, fentanyl, carfentanyl, pentazocine butorphanol, nalbuphine, buprenorphine, sufentanil, alfentanil, tramadol, remifentanil, hydrocodone, oxycodone, hydromorphone, oxymorphone, or similar clinically used opioid receptor agonist drug.

Yet in other embodiments, said adrenoreceptor modulator compound is an α2A adrenergic antagonist. In other embodiments, said adrenoreceptor modulator compound is an opioid receptor modulator.

Still in other embodiments, said adrenoreceptor modulator compound is of the formula:

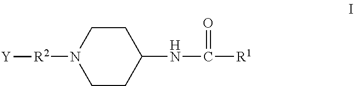

where R¹ is C₁₋₁₀ alkyl, C₁₋₁₀ haloalkyl, optionally substituted aryl, or optionally substituted heteroaryl; R² is C₁₋₆ alkylene; and Y is optionally substituted aryl, optionally substituted heteroaryl, or a moiety of the formula —C(═O)—X¹, wherein X¹ is —OR₃ or —NR⁴R⁵, where each of R³, R⁴ and R⁵ is H or C₁₋₁₀ alkyl. In some embodiments, R¹ is selected from the group consisting of ethyl, 7-bromoheptyl, fur-2-yl, fur-3-yl, and phenyl. Still in other embodiments, R² is ethylene. In further embodiments, Y is selected from the group consisting of phenyl, thiophen-2-yl, and a moiety of the formula —C(═O)—OR₃, where R³ is C₁₋₁₀ alkyl.

In further embodiments, said adrenoreceptor modulator compound comprises N-(1-phenethylpiperidin-4-yl)propionamide (Compound 1), N-(1-phenethylpiperidin-4-yl)furan-2-carboxamide (Compound 2), N-(1-phenethylpiperidin-4-yl)furan-3-carboxamide (Compound 3), N-(1-(2-(thiophen-2-yl)ethyl) piperidin-4-yl)propionamide (Compound 4), or a mixture thereof.

Still in other embodiments, said pain is chronic pain. In other embodiments, said pain is acute pain. Yet in other embodiments, said pain is nociceptive pain. In further embodiments, said pain is neuropathic pain.

Yet another aspect of the invention provides a composition comprising an opioid receptor agonist compound and a second therapeutically active compound, wherein said second therapeutically active compound has an adrenoreceptor modulator activity and an opioid receptor modulating activity.

In some embodiments, said second biologically active compound is of the formula:

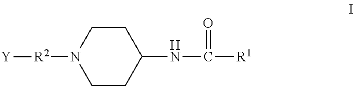

where R¹ is C₁₋₁₀ alkyl, C₁₋₁₀ haloalkyl, optionally substituted aryl, or optionally substituted heteroaryl; R² is C₁₋₆ alkylene; and Y is optionally substituted aryl, optionally substituted heteroaryl, or a moiety of the formula —C(═O)—X¹, wherein X¹ is —OR₃ or —NR⁴R⁵, where each of R³, R⁴ and R⁵ is H or C₁₋₁₀ alkyl. In some instances, R¹ is selected from the group consisting of ethyl, 7-bromoheptyl, fur-2-yl, fur-3-yl, and phenyl. Still in other instances, R² is ethylene. In further instances, Y is selected from the group consisting of phenyl, thiophen-2-yl, and a moiety of the formula —C(═O)—OR₃, where R³ is C₁₋₁₀ alkyl. Still in other embodiments, said second biologically active compound comprises N-(1-phenethylpiperidin-4-yl)propionamide, N-(1-phenethylpiperidin-4-yl)furan-2-carboxamide, N-(1-phenethylpiperidin-4-yl)furan-3-carboxamide, or N-(1-(2-(thiophen-2-yl)ethyl) piperidin-4-yl)propionamide.

Certain piperdinyl-formamide (or similar) compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, adrenoreceptor antagonist compounds encompassed within Formula II are provided:

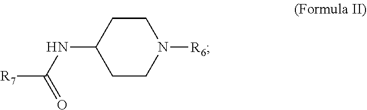

including pharmaceutically acceptable salts, solvates, and/or prodrugs thereof.

Formula II is not limited to a particular chemical moiety for R6 and R7. In some embodiments, the particular chemical moiety for R6 and R7 independently include any chemical moiety that permits the resulting compound to inhibit and/or antagonize adrenoeceptor activity. In some embodiments, the particular chemical moiety for R6 and R7 independently include any chemical moiety that permits the resulting compound to inhibit and/or antagonize opioid receptor activity. In some embodiments, the particular chemical moiety for R6 and R7 independently include any chemical moiety that permits the resulting compound to inhibit and/or antagonize α2A adrenoreceptor activity. In some embodiments, the particular chemical moiety for R6 and R7 independently include any chemical moiety that permits the resulting compound to prevent and/or attenuate the addictive properties of opioid receptor agonist compounds. In some embodiments, the particular chemical moiety for R6 and R7 independently include any chemical moiety that permits the resulting compound to treat, prevent or attenuate pain. In some embodiments, the particular chemical moiety for R6 and R7 independently include any chemical moiety that permits the resulting compound to treat, prevent or attenuate nociceptive pain. In some embodiments, the particular chemical moiety for R6 and R7 independently include any chemical moiety that permits the resulting compound to treat, prevent or attenuate neuropathic pain. In some embodiments, the particular chemical moiety for R6 and R7 independently include any chemical moiety that permits the resulting compound to provide non-addictive pain relief to a subject upon co-administration with an opioid receptor agonist compound. In some embodiments, the particular chemical moiety for R6 and R7 independently include any chemical moiety that permits the resulting compound to provide direct treatment for opioid addiction disorder to a subject upon co-administration with an opioid receptor agonist compound.

In some embodiments, R6 is selected from

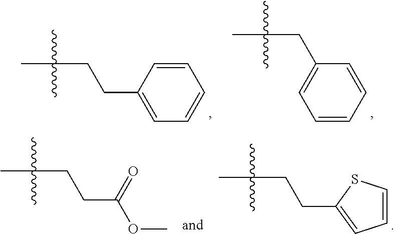

In some embodiments,

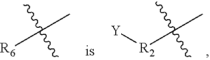

R2 is C₁₋₆ alkylene; Y is optionally substituted aryl, optionally substituted heteroaryl, or a moiety of the formula —C(═O)—X¹; X¹ is —OR₃ or —NR⁴R⁵; and each of R³, R⁴ and R⁵ is independently H or C₁₋₁₀ alkyl.

In some embodiments, R2 is ethylene.

In some embodiments, Y is selected from phenyl, thiophen-2-yl, and a moiety of the formula —C(═O)—OR₃, where R³ is C₁₋₁₀ alkyl.

In some embodiments, R7 is selected from

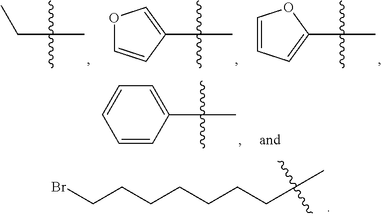

In some embodiments, R7 is selected from C₁₋₁₀ alkyl, C₁₋₁₀ haloalkyl, optionally substituted aryl, and optionally substituted heteroaryl.

In some embodiments, R7 is selected from ethyl, 7-bromoheptyl, fur-2-yl, fur-3-yl, and phenyl.

In some embodiments, the compound is selected from:

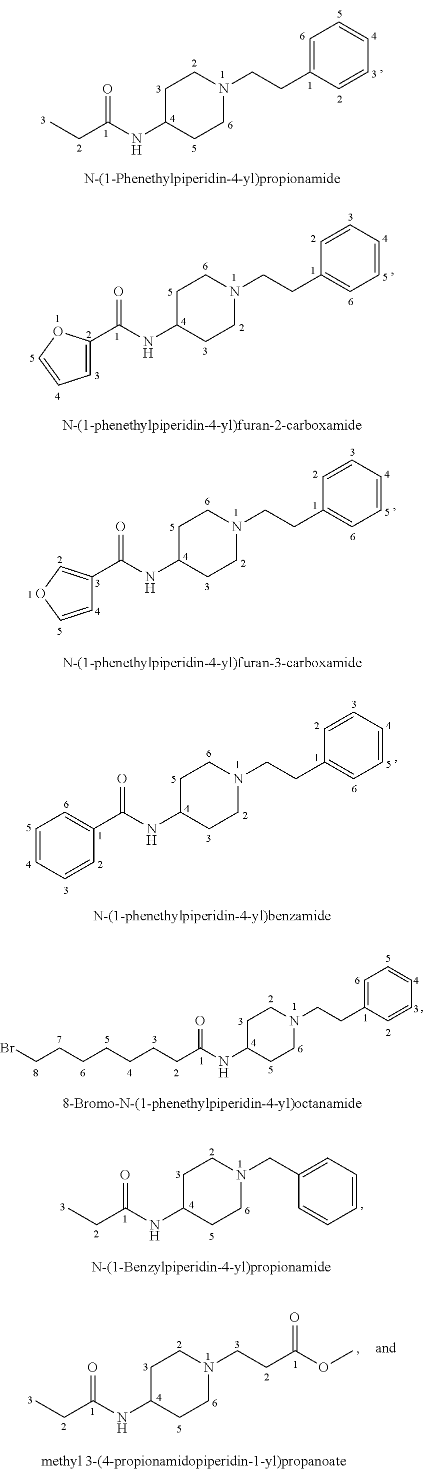

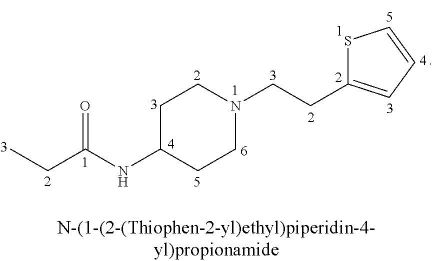 [SMILES: CCC(=O)NC1CCN(CCc2cccs2)CC1]

The adrenoreceptor antagonist compounds described herein can be considered as potential therapeutics for the treatment, prevention, and/or amelioration of conditions characterized with pain (e.g., nociceptive pain; neuropathic pain).

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, conditions related to any type or kind of pain (e.g., nociceptive pain; neuropathic pain).

Some embodiments of the present invention provide methods for administering an effective amount of a compound of the invention and at least one additional therapeutic agent (including, but not limited to, any agent useful in treating pain). In some embodiments, the additional therapeutic agent is one or more opioid receptor agonist compounds (e.g., morphine, fentanyl, carfentanyl, pentazocine, butorphanol, nalbuphine, buprenorphine, sufentanil, alfentanil, tramadol, remifentanil, hydrocodone, oxycodone, hydromorphone, oxymorphone, or similar clinically used opioid receptor agonist drug).

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

The piperdinyl-formamide (or similar) compounds described herein can be considered as potential therapeutics for the treatment, prevention, and/or amelioration of pain (e.g., nociceptive pain; neuropathic pain).

The piperdinyl-formamide (or similar) compounds described herein can be considered as potential therapeutics for the treatment, prevention, and/or amelioration of pain (e.g., nociceptive pain; neuropathic pain) when administered in combination with opioid receptor agonist compounds (e.g., morphine, fentanyl, carfentanyl, pentazocine, butorphanol, nalbuphine, buprenorphine, sufentanil, alfentanil, tramadol, remifentanil, hydrocodone, oxycodone, hydromorphone, oxymorphone, or similar clinically used opioid receptor agonist drug).

The piperdinyl-formamide (or similar) compounds described herein can be considered as potential therapeutics for the treatment, prevention, and/or amelioration of addiction related to opioid receptor agonist compounds.

The piperdinyl-formamide (or similar) compounds described herein can be considered as potential therapeutics for the treatment, prevention, and/or amelioration of conditions associated with adrenoreceptor activity.

The piperdinyl-formamide (or similar) compounds described herein can be considered as potential therapeutics for the treatment, prevention, and/or amelioration of conditions associated with α2A adrenoreceptor activity.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention. As used herein, personal pronouns such as “our”, “we”, “I”, etc. refer to the inventors of the present invention.

### Example I

Synthesis of 1-Phenethylpiperidin-4-one oxime: 1-Phenethylpiperidin-4-one 10.15 g (0.05 mol) dissolved in 60 mL of ethanol was added dropwise at 0° C. to a water solution of hydroxylamine in water (prepared by adding 13.8 g (0.1 mol) of K₂CO₃ to the solution of 6.95 g (0.1 mol) hydroxylamine hydrochloride in 50 mL of water). The mixture was left overnight. Ethanol was evaporated under slight vacuum. Water was added and the mixture was stirred on ice bath for an hour. The separated solid product was filtered, washed with water and dried on the air. The crude oxime 10.71 g (98.25%), m.p. 132-134° C., was used in the next reaction without further purification. MS (ESI): 219.1 (MH+).

Synthesis of 1-Phenethylpiperidin-4-amine: 1-Phenethylpiperidin-4-one oxime 6.54 g (0.03 mol) was dissolved in 100 ml of dry i-AmOH on heating and ten fold excess of sodium 6.9 g (0.3 mol) was slowly added to the stirred solution in small pieces, keeping temperature around 110° C. Solution was stirred at 110° C. for two hours and left to cool to room temperature. The mixture was diluted with 150 ml of ether followed by 75 mL. Organic layer was separated, dried over MgSO₄, and filtered. Solvent was removed and the product was distilled under vacuum to give 4.3 g (70%) of 1-phenethylpiperidin-4-amine. B.P. 138-142° C./1.5 mm. MS (ESI): 205. (MH+).

Synthesis of N-(1-Phenethylpiperidin-4-yl)propionamide: Propionyl chloride 2.775 g (0.03 mol) in 5.55 mL of CHCl₃ was added dropwise to a cooled (0° C.) solution of 4.08 g (0.02 mol) 1-phenethylpiperidin-4-amine and 3.03 g (0.03 mol) of Et₃N in 30 mL of CHCl₃. The mixture was allowed to reach room temperature and stirred overnight. After work-up with 5% aqueous solution of NaHCO₃, organic layer was separated, washed with water, dried over MgSO₄, and filtered. Filtrate was concentrated and crystallized from hexane to give 4.9 g (94%) of N-(1-phenethylpiperidin-4-yl)propionamide. M.p.134-135° C. MS (ESI): 261.2 (MH+). ¹H NMR (600 MHz, CDCl₃): δ 7.27 (t, J=7.4 Hz, 2H), 7.19 (m, 3H), 5.32 (d, J=7.4 Hz, 1H), 3.82 (qt, J=7.8, 4.2 Hz, 1H), 2.92 (dt, J=11.8, 3.4 Hz, 2H), 2.79 (m*, 2H), 2.59 (m*, 2H), 2.19 (q, J=7.5 Hz, 2H), 2.18 (m, 2H), 1.95 (dtd, J=12.4, 4.4, 1.7 Hz, 2H), 1.46 (qd, J=11.7, 3.8 Hz, 2H), 1.15 (t. J=7.5 Hz, 3H). ¹³C NMR (150) MHz, CDCl₃): δ 173.0, 140.2, 128.6, 128.4, 126.0, 60.4, 52.3, 46.3, 33.7, 32.3, 29.8, 9.9.

Synthesis of N-(1-Phenethylpiperidin-4-yl)propionamide oxalate. Oxalic acid 1 g (0.011 mol) in 10 mL of ethanol was added dropwise to the solution of 2.93 g (0.011 mol) of N-(1-phenethylpiperidin-4-yl)propionamide in 29.3 mL of ethanol. The mixture was left overnight. Obtained crystals were separated and dried in dessicator over P₂O₅ to give 3.5 g of N-(1-phenethylpiperidin-4-yl)propionamide oxalate. M.P. 216-218° C. MS (ESI): 261.2 (M+). X-ray crystallography data for a representative compound of the invention, N-(1-phenethyl-piperidin-4-yl)propionamide oxalate confirmed the structure.

Synthesis of N-(1-phenethylpiperidin-4-yl)furan-2-carboxamide. 2-Furoyl chloride 0.827 g. (0.00636 mol) dissolved in 0.25 mL of dry dichloromethane was added dropwise to a cooled ((° C.)) solution of 1.08 g (0.00468 mol) 1-phenethylpiperidin-4-amine and 0.178 mL of Et₃N in 2 mL of CHCl₃. The mixture was allowed to reach room temperature and stirred overnight. After work-up with 5% solution of NaHCO₃, organic layer was separated, washed with water, dried over MgSO₄, and filtered. Filtrated was concentrated to yield a white solid. The product was washed with hexanes to obtain an analytically pure sample. Yield: 0.0845 g (58.3%). MS (ESI): 299.3 (MH+). ¹H NMR (400 MHZ, CDCl₃): δ 1.63 (d, J=11.93 Hz, 2H), 2.05 (d, J=12.33 Hz, 2H), 2.26 (t, J=11.35, 11.35 Hz, 2H), 2.64 (dd, J=6.16, 10.21 Hz, 2H), 2.83 (dd, J=6.12, 10.24 Hz, 2H), 2.99 (d, J=12.01 Hz, 2H), 3.98 (m, 1H), 6.21 (d, J=8.20 Hz, 1H), 6.49 (dd, J=1.79, 3.47 Hz, 1H), 7.10 (dd, J=0.84, 3.47 Hz, 1H), 7.24 (m, 5H), 7.43 (dd, 0.84, 1.77 Hz, 1H). ¹³C NMR (100 MHZ, CDCl₃): δ 32.67, 34.17, 46.27, 46.61, 52.76, 60.88, 112.60, 114.57, 126.56, 128.87, 129.13, 140.58, 144.16, 148.48, 158.13. Obtained compound was transformed to oxalate salt as described above.

f Synthesis of N-(1-phenethylpiperidin-4-yl)furan-3-carboxamide. A solution of 3-furoyl chloride 0.827 g. (0.00636 mol) in 0.25 mL of dry dichloromethane was added dropwise to a mixture of cooled (0° C.)) solution of 1.08 g (0.00468 mol) 1-phenethyl-piperidin-4-amine and 0.178 mL of Et₃N in 2 mL of CHCl₃. The mixture was left to reach room temperature and stirred overnight. After work-up with 5% solution of NaHCO₃, organic layer was separated, washed with water, dried over MgSO₄, and filtered. Filtrate was concentrated to yield a white solid. The product was washed with hexanes to obtain an analytically pure sample. Yield: 0.104 g (72%). MS (ESI): 299.3 (MH+). ¹H NMR (400 MHZ, CDCl₃): δ 1.63 (m, 2H), 2.06 (d, J=11.35 Hz, 2H), 2.26 (m, J=11.35, 2H), 2.65 (m, 2H), 2.84 (m, 2H), 3.02 (d, J=11.86 Hz, 2H), 3.99 (m, 1H), 5.65 (d. J=7.99 Hz, 1H), 6.59 (dd, J=0.91, 1.93 Hz, 1H), 7.24 (m, 5H), 7.42 (dd, 1.58, 1.91 Hz, 1H), 7.91 (dd, J=0.90, 1.59 Hz, 1H). ¹³C NMR (100 MHZ, CDCl₃): δ 32.04, 33.49, 46.43, 52.35, 60.26, 108.25, 122.66, 126.19, 128.46, 128.68, 139.88, 143.72, 144.69, 161.95. Obtained compound was transformed to oxalate salt as described above.

Synthesis of N-(1-phenethylpiperidin-4-yl)benzamide. A solution of benzoyl chloride 0.89 g. (0.00636 mol) in 0.25 mL of dry dichloromethane was added dropwise to a cooled (0° C.) solution of 1.08 g (0.00468 mol) 1-phenethylpiperidin-4-amine and 0.178 mL of Et₃N in 2 mL of CHCl₃. The mixture was allowed to reach to room temperature and stirred for a night. After work-up with 5% solution of NaHCO₃, organic layer was separated, washed with water, dried over MgSO₄, and filtered. Concentration of the filtrate gave a white solid. The product was washed with hexanes to obtain an analytically pure sample. Yield: 0.132 g (87%). MS (ESI): 309.2 (MH+). ¹H NMR (400 MHZ, CDCl₃): δ 1.35 (t, J=7.29, 7.29 Hz, 2H), 1.64 (qd, J=3.80, 11.29, 11.29, 11.35 Hz, 2H), 2.08 (m, 2H), 2.27 (td, J=2.57, 11.61, 11.65 Hz, 2H), 2.64 (m, 2H), 2.83 (m, 2H), 3.00 (m, 3H), 4.04 (dddd, J=4.28, 8.29, 10.85, 15.24 Hz, 1H), 6.04 (d, J=7.94 Hz, 1H), 7.25 (m, 5H), 7.44 (m, 3H), 7.75 (m, 2H). ¹³C NMR (100 MHZ, CDCl₃): δ 32.22, 33.71, 45.83, 46.97, 52.38, 60.42, 114.25, 126.12, 126.86, 128.42, 128.56, 128.68, 131.42, 134.75, 140.11, 166.88. Obtained compound was transformed to oxalate salt as described above.

Synthesis of 8-Bromo-N-(1-phenethylpiperidin-4-yl)octanamide: A solution of 1-phenethylpiperidin-4-amine (0.101 g, 0.00493 mol), 8-bromooctanoic acid (0.1 g, 0.00448 mol), HATU (0.170 g, 0.00448 mol), HOAt (0.061 g, 0.00448 mol), and DIEPA (0.314 mL, 0.0018 mol) in dry DMF was stirred at room temperature overnight. The reaction mixture was then quenched with 0.5 M KHSO₄ solution followed by the addition of dichloromethane. The organic and aqueous layers were separated, and the aqueous layer was extracted with dichloromethane (3×5 mL) followed by washing with NaHCO₃ solution and brine. The organic extracts were combined, dried over anhydrous magnesium sulfate, and filtered. Concentration of the filtrate gave a white solid. Yield: 0.146 g (80%). MS (ESI): 409.2 (MH+). ¹H NMR (400 MHZ, CDCl₃): δ 1.32-2.34 (m, 14H), 2.82-3.01 (m, 11H), 3.16 (dd, J=6.53, 10.91 Hz, 1H), 3.49 (d, J=11.91 Hz, 1H), 4.63 (dt, J=5.61, 5.61, 8.76 Hz, 1H), 7.24 (m, 4H), 7.42 (dd, J=4.46, 8.37, 1H), 8.02 (m, 1H). ¹³C NMR (100 MHZ, CDCl₃): δ 25.17, 25.45, 27.85, 28.70, 28.75, 31.46, 36.55, 38.61, 81.47, 120.73, 128.66, 128.86, 129.19, 151.29, 162.71, 173.62. Obtained compound was transformed to oxalate salt as described above.

Synthesis of 1-Benzylpiperidin-4-one oxime: A solution of 1-benzylpiperidin-4-one (28.35 g, 0.15 mol) in 60 mL of EtOH was added to the cooled to 0° C.) solution of hydroxylamine hydrochloride (20.85 g, 0.30 mol) in 75 mL of H₂O. To the resulting mixture was added a solution of K₂CO₃ (20.7 g, 0.15 mol) in 75 mL of H₂O. The reaction mixture was then allowed to reach room temperature and stirred overnight. The EtOH was removed and the reaction mixture was cooled in an ice bath to allow the product to crystallize out of solution. The product was filtered and washed several times with H₂O and recrystallized in EtOH. Yield: 27.78 g (70.17%).

Synthesis of 1-Benzylpiperidin-4-amine: Na metal (6.9 g, 0.3 mol) was added to a 110° C. solution of 1-benzylpiperidin-4-one oxime (6.12 g, 0.03 mol) in 90 mL of iso-amyl alcohol. The reaction mixture was allowed to cool to room temperature and stirred until the reaction mixture became a thick slurry. The slurry was dissolved in 50 mL of diethyl ether and 25 mL of H₂O. The organic layer was separated, washed with H₂O, dried over anhydrous magnesium sulfate, and filtered. The filtrate was concentrated to yield a yellow oil. The crude product was purified via column chromatography using silica gel and a DCM:MeOH solvent in a ratio of 4:1 with an additional 1% of Et₃N. Yield: 3.7 g (64%).

Synthesis of N-(1-Benzylpiperidin-4-yl)propionamide. To a 0° C.) solution of 1-benzylpiperidin-4-amine (3.7 g, 0.019 mol) and 5 mL of Et₃N (0.05 mol) in 45 mL of dry dichloromethane was added a solution of propionyl chloride (2.17 mL, 0.025 mol) in 10 mL of dichloromethane. The reaction mixture was allowed to reach room temperature and stirred overnight. To the reaction mixture was added 4 mL of NH₄OH and 45 mL of H₂O. The aqueous layer was separated and extracted with dichloromethane. The combined organic layers were washed with NaHCO₃ solution and brine, dried over anhydrous magnesium sulfate, filtered and concentrated to yield a white solid. The product was washed with hexanes to obtain an analytically pure sample. Yield: 2.8 g (72%)

Synthesis of N-(Piperidin-4-yl)propionamide: A solution of N-(1-benzylpi-peridin-4-yl)propionamide (0.7 g, 0.003 mol) in 30 mL of EtOH was hydrogenated for 24 hours under 50 psi of H₂ in the presence of 10% Pd/C (0.07 g) and 20% Pd(OH)₂ (0.07 g). The solution was filtered through celite and the solvent was evaporated to yield 0.467 g (99%) of the product.

Synthesis of methyl 3-(4-propionamidopiperidin-1-yl) propanoate. A solution of N-(piperidin-4-yl)propionamide (0.1 g, 0.0052 mol) in 2 mL of dry acetonitrile and methyl acrylate (0.071 mL, 0.00789 mol) was refluxed overnight. The solvent was removed and the crude product was purified by washing with hexanes to yield 0.90 g (71%) of the product. MS (ESI): 243.3 (MH+). ¹H NMR (400 MHZ, CDCl₃): δ 1.14 (t, J=7.59, 7.59 Hz, 3H), 1.41 (dtd, J=3.70, 11.10, 11.13, 12.61 Hz, 2H), 1.91 (m, 2H), 2.17 (m, 4H), 2.49 (m, 2H), 2.68 (m, 2H), 2.81 (m, 2H), 3.67 (s, 3H), 3.78 (dddd, J=4.29, 4.36, 11.92, 15.26, 1H), 5.25 (d, J=7.96 Hz, 1H). ¹³C NMR (100 MHZ, CDCl₃): δ 10.02, 29.99, 32.41, 46.40, 51.76, 52.25, 55.63, 173.14, 174.05. Obtained compound was transformed to oxalate salt as described above.

Synthesis of N-(1-(2-(Thiophen-2-yl)ethyl) piperidin-4-yl)propionamide. A solution of N-(piperidin-4-yl)propionamide (0.1 g, 0.0064 mol), 2-(thiophen-2-yl)ethyl methanesulfonate (0.145 g, 0.704 mol), K₂CO₃ (0.097 g, 0.00704 mol), KI (0.032 g, 0.00192 mol), Et₃N (0.178 mL, 0.00128 mol) in 5 mL of dry acetonitrile was stirred overnight under refluxing conditions. The mixture was concentrated, and the residue was diluted with H₂O and extracted with ethyl acetate. The organic extracts were combined, dried over anhydrous magnesium sulfate, filtered and concentrated. The crude product was washed with hexanes to obtain an analytically pure sample. Yield: 0.101 g (60%). MS (ESI): 267.7 (MH+). ¹H NMR (400 MHZ, CDCl₃) δ 1.15 (t, J=7.58, 7.58 Hz, 3H), 1.47 (m, 2H), 1.95 (m, 2H), 2.18 (m, 4H), 2.64 (dd, J=6.87, 8.62 Hz, 2H), 2.90 (m, 2H), 3.00 (m, 2H), 3.82 (dddd, J=4.20, 8.31, 10.86, 15.17 Hz, 1H), 5.32 (d, J=7.95 Hz, 1H), 6.81 (dq, J=1.02, 1.02, 1.02, 3.20 Hz, 1H), 6.91 (dd, J=3.39, 5.14 Hz, 1H), 7.11 (dd, J=1.21, 5.13 Hz, 1H). ¹³C NMR (100 MHZ, CDCl₃) δ 10.04, 28.06, 30.03, 32.15, 46.52, 52.42, 59.98, 123.62, 124.71, 126.69, 142.87, 173.15. Obtained compound was transformed to oxalate salt as described above.

Bioavailability and Stability: Compounds of the invention were studied in detail, using several parameters to evaluate bioavailability and stability. In summary, Compound 1 was highly bioavailable, indicating that it is likely that this compound is highly bioavailable when given by the oral route of administration.

Toxicity: The Compound 1 was studied for its toxicity in many animal models. Compound 1 showed virtually no toxicity even at high concentrations.

In vivo test: Compound 1 showed significant inhibition of L-DOPA induced dyskinesias in both the Rodent and Primate in vivo models. Compound 1 was significantly more effective than amantadine which is the gold standard therapy for patients with Parkinson's disease to reduce the effects of L-dopa-induced dyskinesia.

Cytochrome P450 Inhibition: The objective was to access the potential of Compound 1 to inhibit the main cytochrome P450 isoforms, CYP1A2, CYP2B6, CYP2C8, CYP2C9, CYP2C19, CYP2D6 and CYP3A4 (2 substrates). Compound 1 (0.1 μM-25 μM) was incubated with human liver microsomes and NADPH in the presence of a cytochrome P450 isoform-specific probe substrate. The metabolites were monitored by mass spectrometry. A decrease in the formation of the metabolite compared to the vehicle control is used to calculate an IC₅₀ value for each P450 (concentration of Compound 1 which produces 50% inhibition).

CYP1A2 Inhibition: Six test compound concentrations (0.1, 0.25, 1, 2.5, 10, 25 μM in DMSO; final DMSO concentration 0.3%) were incubated with human liver microsomes (0.25 mg/mL) and NADPH (1 mM) in the presence of the probe substrate phenacetin (30 μM) for 5 min at 37° C. The selective CYP1A2 inhibitor, α-naphthoflavone, was screened alongside Compound 1 as a positive control.

CYP2B6 Inhibition: Six test compound concentrations (0.1, 0.25, 1, 2.5, 10, 25 μM in DMSO; final DMSO concentration 0.3%) were incubated with human liver microsomes (0.1 mg/mL) and NADPH (1 mM) in the presence of the probe substrate bupropion (110 μM) for 5 min at 37° C. The selective CYP2B6 inhibitor, ticlopidine, was screened alongside Compound 1 as a positive control.

CYP2C8 Inhibition: Six test compound concentrations (0.1, 0.25, 1, 2.5, 10, 25 μM in DMSO; final DMSO concentration 0.3%) were incubated with human liver microsomes (0.25 mg/mL) and NADPH (1 mM) in the presence of the probe substrate paclitaxel (7.5 μM) for 30 min at 37° C. The selective CYP2C8 inhibitor, montelukast, was screened alongside Compound 1 as a positive control.

CYP2C9 Inhibition: Six test compound concentrations (0.1, 0.25, 1, 2.5, 10, 25 μM in DMSO; final DMSO concentration 0.25%) were incubated with human liver microsomes (1 mg/mL) and NADPH (1 mM) in the presence of the probe substrate tolbutamide (120 μM) for 60 min at 37° C. The selective CYP2C9 inhibitor, sulphaphenazole, was screened alongside Compound 1 as a positive control.

CYP2C19 Inhibition: Six test compound concentrations (0.1, 0.25, 1, 2.5, 10, 25 μM in DMSO; final DMSO concentration 0.25%) were incubated with human liver microsomes (0.5 mg/mL) and NADPH (1 mM) in the presence of the probe substrate mephenytoin (25 μM) for 60 min at 37° C. The selective CYP2C19 inhibitor, tranylcypromine, was screened alongside Compound 1 as a positive control.

CYP2D6 Inhibition: Six test compound concentrations (0.1, 0.25, 1, 2.5, 10, 25 μM in DMSO; final DMSO concentration 0.25%) were incubated with human liver microsomes (0.5 mg/mL) and NADPH (1 mM) in the presence of the probe substrate dextromethorphan (5 μM) for 5 min at 37° C. The selective CYP2D6 inhibitor, quinidine, was screened alongside Compound 1 as a positive control.

CYP3A4 Inhibition (Midazolam): Six test compound concentrations (0.1, 0.25, 1, 2.5, 10, 25 μM in DMSO; final DMSO concentration 0.26%) were incubated with human liver microsomes (0.1 mg/mL) and NADPH (1 mM) in the presence of the probe substrate midazolam (2.5 μM) for 5 min at 37° C. The selective CYP3A4 inhibitor, ketoconazole, was screened alongside Compound 1 as a positive control.

CYP3A4 Inhibition (Testosterone): Six test compound concentrations (0.1, 0.25, 1, 2.5, 10, 25 μM in DMSO; final DMSO concentration 0.275%) were incubated with human liver microsomes (0.5 mg/mL) and NADPH (1 mM) in the presence of the probe substrate testosterone (50 μM) for 5 min at 37° C. The selective CYP3A4 inhibitor, ketoconazole, was screened alongside Compound 1 as a positive control.

The reactions were terminated by methanol. The samples were then centrifuged and the supernatants were analyzed for acetaminophen, hydroxy bupropion, 6α-hydroxypaclitaxel 4-hydroxytolbutamide, 4-hydroxymephenytoin, dextrorphan, 1-hydroxymidazolam and 6β-hydroxytestosterone by LC-MS/MS. Formic acid in deionized water (final concentration 0.1%) containing internal standard is added to the final sample prior to analysis. A decrease in the formation of the metabolites compared to vehicle control was used to calculate an IC₅₀ value (concentration of Compound 1 which produces 50% inhibition).

Results: Out of the 7 CYPs tested, Compound 1 was only found to significantly inhibit CYP2D6, and weakly CYP2C19. With CYP2C19, the inhibition was too weak to generate an IC₅₀ value, and 36.4% inhibition was observed at the top concentration of 25 micromolar. With CYP2D6, an IC₅₀ of 4.2 micromolar was obtained. No significant inhibition was observed at CYP2B6, CYP2C9, CYP3A4 (with either substrate), CYP2C8 or CYP1A2.

hERG Channel Inhibition (IC₅₀ Determination): Mammalian cells expressing the hERG potassium channel were dispensed into 384-well planar arrays and hERG tail-currents measured by whole-cell voltage-clamping. A range of concentrations of the test compound was then added to the cells and a second recording of the hERG current was made. The percent change in hERG current was calculated and used to calculate an IC₅₀ value (test compound concentration which produces 50% inhibition).

The experiments were performed on an IonWorks™ automated patch clamp instrument (Molecular Devices LLC), which simultaneously performs electrophysiology measurements for 48 single cells in a specialized 384-well plate (PatchPlate™). All cell suspensions, buffers and test compound solutions were at room temperature during the experiment. The cells used were Chinese hamster ovary (CHO) cells stably transfected with hERG (cell-line obtained from Cytomyx, UK). A single-cell suspension was prepared in extracellular solution (Dulbecco's phosphate buffered saline with calcium and magnesium pH 7.2) and aliquots were added automatically to each well of a PatchPlate™. The cells were then positioned over a small hole at the bottom of each well by applying a vacuum beneath the plate to form an electrical seal. The vacuum was applied through a single compartment common to all wells which was filled with intracellular solution (buffered to pH 7.2 with HEPES). The resistance of each seal was measured via a common ground-electrode in the intracellular compartment and individual electrodes placed into each of the upper wells.

Electrical access to the cell was then achieved by circulating a perforating agent, amphotericin B, underneath the PatchPlate™. The pre-compound hERG current was then measured. An electrode was positioned in the extracellular compartment and a holding potential of −80 mV applied for 15 sec. The hERG channels were then activated by applying a depolarizing step to +40 mV for 5 sec and then clamped at −50 mV for 4 sec to elicit the hERG tail current, before returning to −80 mV for 0.3 sec. Compound dilutions were prepared by diluting a DMSO solution (default 10 mM) of the test compound using a factor 5 dilution scheme into DMSO, followed by dilution into extracellular buffer such that the final concentrations tested were typically 0.008, 0.04, 0.2, 1, 5, 25 μM (final DMSO concentration 0.25%). The IonWorks™ instrument automatically added test compound dilutions to the upper wells of the PatchPlate™. The test compound was left in contact with the cells for 300 sec before recording currents using the same voltage-step protocol as in the pre-compound scan. Quinidine, an established hERG inhibitor, was included as a positive control, and vehicle control (0.25% DMSO) as negative control.

Each concentration is tested in 4 replicate wells on the PatchPlate™ (maximum of 32 data points). Filters were applied to ensure only acceptable cells were used to assess hERG inhibition.

Results: Over the concentration range tested (up to 25 micromolar) no dose-response was obtained, therefore the inhibition IC50 is >25 micromolar. There was a hint of some inhibition at the top concentration of 25 micromolar, with 32.5% inhibition observed (insufficient to generate an IC50 value). The results showed the compound had weak or no significant hERG inhibition activity.

MDR1-MDCK Permeability and Identification of P-gp Substrate (Bi-directional): Madin-Darby canine kidney (MDCK) cells are an epithelial cell line of canine kidney origin. These cells can be stably transfected to express active P-glycoprotein (MDR1-MDCK) and are ideal for studying drug efflux. Test compound was added to either the apical or basolateral side of a confluent monolayer of MDR1-MDCK cells and permeability was measured by monitoring the appearance of the test compound on the opposite side of the monolayer using LC-MS/MS. The efflux ratio (ER) was calculated from the ratio of B-A and A-B permeabilities. Experiments were performed in the absence and presence of a P-glycoprotein (P-gp) inhibitor (cyclosporin A (10 μM)) to determine whether the compound was subject to P-gp mediated efflux.

Experimental Procedure: MDR1-MDCK cells obtained from the NIH (Rockville, MD, USA) were used between passage numbers 6-30. Cells were seeded onto Millipore Multiscreen Transwell plates at 3.4×10⁵ cells/cm². The cells were cultured in DMEM and media was changed on day 3. On day 4 the permeability study was performed. Cell culture and assay incubations were carried out at 37° C. in an atmosphere of 5% CO₂ with a relative humidity of 95%. On the day of the assay, the monolayers were prepared by rinsing both apical and basolateral surfaces twice with Hanks Balanced Salt Solution (HBSS) at the desired pH warmed to 37° C. Cells were then incubated with HBSS at the desired pH in both apical and basolateral compartments for 40 min to stabilize physiological parameters.

The dosing solutions were prepared by diluting test compound with assay buffer to give a final test compound concentration of 10 μM (final DMSO concentration of 1% v/v). The fluorescent integrity marker lucifer yellow was also included in the dosing solution. Where applicable, the P-gp inhibitor was also included. Analytical standards were prepared from test compound DMSO dilutions and transferred to buffer, maintaining a 1% v/v DMSO concentration. Test compound permeability was measured on two occasions, in the absence and in the presence of a P-gp inhibitor on both sides of the monolayer. The assay buffer was composed of supplemented HBSS pH 7.4.

For assessment of A-B permeability. HBSS was removed from the apical compartment and replaced with test compound dosing solution. The apical compartment insert was then placed into a companion plate containing fresh buffer (containing 1% v/v DMSO or, where applicable, a P-gp inhibitor, maintaining a 1% v/v DMSO concentration). For assessment of B-A permeability. HBSS was removed from the companion plate and replaced with test compound dosing solution. Fresh buffer (containing 1% v/v DMSO or, where applicable, a P-gp inhibitor, maintaining a 1% v/v DMSO concentration) was added to the apical compartment insert, which was then placed into the companion plate. At 60 min the apical compartment inserts and the companion plates were separated and apical and basolateral samples diluted for analysis. Test compound permeability was assessed in duplicate. Compounds of known permeability characteristics were run as controls on each assay plate. Test and control compounds were quantified by LC-MS/MS cassette analysis using an 8-point calibration with appropriate dilution of the samples. Cyprotex generic analytical conditions were used. The starting concentration (C₀) was determined from the dosing solution and the experimental recovery calculated from C₀ and both apical and basolateral compartment concentrations.

The integrity of the monolayer throughout the experiment was checked by monitoring lucifer yellow permeation using fluorimetric analysis. Lucifer yellow permeation was high if monolayers have been damaged. If a lucifer yellow Pₐₚₚ value was above a pre-defined threshold in one individual test compound well, the compound was re-tested or an n=1 result was reported. If lucifer yellow Pₐₚₚ values were above the threshold in both replicate wells for a test compound, the compound was re-tested. If this re-occurred upon repeat in both wells then toxicity or inherent fluorescence of the test compound was assumed. No further experiments were performed in this instance.

Results: Compound 1 was highly permeable in the MDCK assay. There was a slight difference between the plus and minus inhibitor data in terms of the efflux ratio obtained (1.48 minus inhibitor, versus 0.929 plus inhibitor). Typically, a ratio of greater than 2 is indicative that efflux is occurring. The control compounds behaved as expected, with prazosin (a P-gp substrate) showing efflux in the absence of Cyclosporin A, which was inhibited in its presence.

Cytochrome P450 Induction (Cryopreserved Hepatocytes, mRNA Assessment): Experiments were conducted to assess the potential of Compound 1 to induce the cytochrome P450 isoforms CYP1A2, CYP2B6 and CYP3A4 using an mRNA endpoint in cryopreserved hepatocytes.

Experimental Procedure: Cryopreserved human hepatocytes from a single donor were seeded on a 96-well collagen coated plate so that the final seeding density is 0.1×10⁶ cells/well (final volume per well 0.1 mL). The cells were then incubated in seeding medium at 37° C. 95% humidity, 5% CO₂ to allow the cells to attach. After 4 hr, the seeding medium was replaced with 0.1 mL of pre-warmed serum-free Williams E medium (William's E containing 100 IU/mL penicillin, 100 μg/mL streptomycin, 10 μg/mL insulin, 2 mM L-glutamine and 0.1 μM hydrocortisone). The next day, cells were dosed with test compound in assay medium (six final test compound concentrations between 100 μM and 0.4 μM: final DMSO concentration 0.1%). Positive control inducers, omeprazole (100 μM) for CYP1A2, phenobarbital (1000 μM) for CYP2B6 and rifampicin (25 μM) for CYP3A4, were incubated alongside the test compound. Negative control wells were included where the test compound is replaced by vehicle solvent (typically 0.1% DMSO in assay medium). Each test or control compound was dosed in triplicate at each concentration. The cells were exposed to the solutions for 72 hr with fresh solution added every 24 hr.

For mRNA assessment, all media was removed from each of the wells and the cells were washed once with 0.1 mL of pre-warmed assay medium. The cells were lysed by adding 100 μL of lysis buffer to each well. Total RNA was then isolated from the hepatocyte lysates. Reverse transcription was performed and quantitative PCR analysis was performed on the resulting cDNA, using gene-specific primer probe sets for CYP1A2, CYP2B6 and CYP3A4 target cDNA and endogenous control. Samples were analyzed using an ABI 7900 HT real time PCR system.

Data Analysis: For mRNA assessment, relative fold mRNA expression was determined based on the threshold cycle (C_{T}) data of target gene relative to endogenous control for each reaction, and normalized to negative control using the 2^{-ΔΔCT} method. To determine the statistical significance of any fold change of mRNA expression, a one way ANOVA with two tailed Dunnett's post-test was performed using the ΔC_{T} values. Differences with a p value less than 0.05 were taken to be significant.

Results: To summarize, Compound 1 didn't cause a statistically significant change in CYP1A2 or CYP3A4 mRNA expression at any concentration tested. At 100 μM a slight, yet statistically significant decrease in mRNA expression with CYP2B6 was observed. Therefore, no CYP induction was observed, but potentially some down-regulation of CYP2B6 at the highest concentration tested. The positive and negative control compounds included alongside Compound 1 responded as expected.

Caco-2 Permeability (A-B or Bi-directional): Caco-2 cells were used as an in vitro model of the human intestinal epithelium and permit assessment of the intestinal permeability of potential drugs.

Experimental Procedure: Caco-2 cells obtained from the ATCC were used between passage numbers 40-60. Cells were seeded onto Millipore Multiscreen Transwell plates at 1×10⁵ cells/cm². The cells were cultured in DMEM and media was changed every two or three days. On day 20 the permeability study was performed. Cell culture and assay incubations were carried out at 37° C. in an atmosphere of 5% CO₂ with a relative humidity of 95%. On the day of the assay, the monolayers were prepared by rinsing both apical and basolateral surfaces twice with Hanks Balanced Salt Solution (HBSS) at the desired pH warmed to 37° C. Cells were then incubated with HBSS at the desired pH in both apical and basolateral compartments for 40 min to stabilize physiological parameters. The dosing solutions were prepared by diluting test compound with assay buffer to give a final test compound concentration of 10 μM (final DMSO concentration of 1% v/v). The fluorescent integrity marker lucifer yellow was also included in the dosing solution. Analytical standards were prepared from test compound DMSO dilutions and transferred to buffer, maintaining a 1% v/v DMSO concentration.

For assessment of A-B permeability, HBSS was removed from the apical compartment and replaced with test compound dosing solution. The apical compartment insert was then placed into a companion plate containing fresh buffer (containing 1% v/v DMSO). For assessment of B-A permeability, HBSS was removed from the companion plate and replaced with test compound dosing solution. Fresh buffer (containing 1% v/v DMSO) was added to the apical compartment insert, which as then placed into the companion plate. At 120 min the apical compartment inserts and the companion plates were separated and apical and basolateral samples diluted for analysis.

Test compound permeability was assessed in duplicate. Compounds of known permeability characteristics were run as controls on each assay plate. Test and control compounds were quantified by LC-MS/MS cassette analysis using an 8-point calibration with appropriate dilution of the samples. Cyprotex generic analytical conditions were used. The starting concentration (C₀) was determined from the dosing solution and the experimental recovery calculated from C₀ and both apical and basolateral compartment concentrations. The integrity of the monolayer throughout the experiment was checked by monitoring lucifer yellow permeation using fluorimetric analysis. Lucifer yellow permeation is high if monolayers have been damaged. If a lucifer yellow Pₐₚₚ value was above a pre-defined threshold in one individual test compound well, the compound was re-tested or an n=1 result was reported. If lucifer yellow Pₐₚₚ values were above the threshold in both replicate wells for a test compound, the compound was re-tested. If this re-occurred upon repeat in both wells then toxicity or inherent fluorescence of the test compound was assumed. No further experiments were performed in this instance.

Results: The compound was highly permeable in both directions in the Caco-2 assay with an efflux ratio of 2.70. Without being bound by any theory, this is likely due to the compound carrying a basic functionality and is highly unlikely to indicate active transport. The assay has been run with a pH gradient (pH 6.8/7.4) to mimic the intestinal pH.

Time Dependent Inhibition of CYP2D6 (Dextromethorphan; IC₅₀ Shift): Six test compound concentrations (0.1, 0.25, 1, 2.5, 10, 25 μM: final DMSO concentration 0.25%, final microsome concentration 0.5 mg/mL) were either pre-incubated for 30 min in the absence and presence of NADPH or undergo a 0) min pre-incubation. The probe substrate dextromethorphan (5 μM) and NADPH (1 mM) were then added (final DMSO concentration 0.3%) and the samples incubated for 5 min at 37° C. The selective time dependent CYP2D6 inhibitor, paroxetine, was screened alongside the test compounds as a positive control. The reactions were terminated by the addition of an aliquot of the incubation into methanol. The samples were centrifuged at 2500 rpm for 30 min at 4° C., and aliquots of the supernatant are diluted with formic acid in deionised water (final concentration 0.1%) containing internal standard prior to analysis of dextrorphan by LC-MS/MS.

Results: The CYP time-dependent inhibition data was weaker than previously observed. In this assay the IC₅₀ was likely to be just over 25 μM, as approximately 40% inhibition was observed at this concentration (the 0 minute incubation). In the 30 minute plus NADPH incubation (the condition where we would expect to see time-dependent mechanism based inhibition) the percentage inhibition was the same, again at approximately 40%. As such, there is no evidence of time-dependent CYP2D6 inhibition with this compound.

Brain Tissue Binding: Experiments were conducted to measure the extent to which a compound binds to rat or mouse brain homogenate. Solutions of test compound (5 μM, 0.5% final DMSO concentration) were prepared in species specific brain homogenate (1 in 9 dilution in buffer) and in buffer. The experiment was performed using equilibrium dialysis with the two compartments separated by a semi-permeable membrane. The buffer solution was added to one side of the membrane and the brain homogenate solution to the other side. The system was allowed to reach equilibrium over 2 hr at 37° C. Standards were prepared in brain homogenate and buffer and were incubated at 37° C. during the equilibration period. After equilibration, samples were taken from both sides of the membrane. The solutions for each batch of compounds were combined into two groups (buffer and brain homogenate) then cassette analyzed by LC-MS/MS using two sets of calibration standards for buffer (7 points) and brain homogenate solutions (6 points). Cyprotex generic LC-MS/MS conditions ere used. Samples were quantified using standard curves prepared in the equivalent matrix. The compounds were tested in duplicate. Diazepam is included as a control compound in each experiment.

Results: The compound showed weak, or no noticeable binding to plasma proteins and there were very small species differences. In mouse no binding at all could be detected, therefore the fraction unbound could not be determined. In rat brain homogenate low binding was observed, with an fraction unbound of 0.249 reported. Therefore, the compound has a preference for binding brain tissue over plasma proteins.

In Vitro Pharmacology Binding Assays: Representative compounds were tested to determine IC₅₀ or EC₅₀ in receptor functional assays. One or more of the procedures disclosed in the following references were used; Devedjian. J. C. et al., Eur. J. Pharmacol., 1994, 252, 43-49; Wang. J. B. et al., FEBS Lett., 1994, 338, 217-222; Simonin. F. et al., Mol. Pharmacol., 1994, 46, 1015-1021; Meng. F. et al., Proc. Natl. Acad. Sci. U.S.A., 1993, 90, 9954-9958; Avidor-Reiss. T. et al., FEBS Lett., 1995, 361, 70-74; Eason. M. G. et al., J. Biol. Chem., 1992, 267, 15795-15801; Law. P. Y. et al., Mol. Pharmacol., 1993, 43, 684-693. In each experiment and if applicable, the respective reference compound was tested concurrently with the test compounds. Compound binding was calculated as a % inhibition of the binding of a radioactively labeled ligand specific for each target. Cellular agonist effect was calculated as a % of control response to a known reference agonist for each target and cellular antagonist effect was calculated as a % inhibition of control reference agonist response for each target. Only the calculable IC50 and EC50 are reported below.

### Methyl 3-(4-propionamidopiperidin-1-yl) propanoate

| Assay | IC₅₀ | Kᵢ | EC₅₀ | nH |
|---|---|---|---|---|
| α_{2B}(h) (antagonist radioligand) | 2.6E−04M | 1.8E−04M | | 1 |
| κ (KOP) (agonist radioligand) | 4.0E−04M | 2.7E−04M | | 0.8 |
| μ (MOP) (h) (agonist effect) | | | 2.9E−04M | |
| μ (MOP) (h) (agonist radioligand) | 2.0E−04M | 8.2E−05M | | 0.5 |

### N-(1-phenethylpiperidin-4-yl)furan-2-carboxamide

| Assay | IC₅₀ | Kᵢ | K_{B} | EC₅₀ | nH |
|---|---|---|---|---|---|
| α_{2B}(h) (antagonist effect) | 3.8E−04M | | 5.0E−05M | | |
| α_{2B}(h) (antagonist radioligand) | 4.6E−06M | 3.0E−06M | | | 0.7 |
| δ₂ (DOP) (antagonist effect) | 1.4E−05M | | 2.4E−06M | | |
| δ₂ (DOP) (h) (agonist | 9.7E−05M | 5.8E−05M | | | 1 |
| radioligand) | | | | | |
| κ (KOP) (agonist effect) | | | | 5.6E−04M | |
| κ (KOP) (agonist radioligand) | 1.8E−05M | 1.2E−05M | | | 1.1 |
| μ (MOP) (h) (agonist | 2.6E−07M | 1.1E−07M | | | 0.8 |
| radioligand) | | | | | |
| μ (MOP) (h) (antagonist effect) | 1.4E−05M | | 1.5E−06M | | |

### N-(1-phenethylpiperidin-4-yl)furan-3-carboxamide

| Assay | IC₅₀ | Kᵢ | K_{B} | EC₅₀ | nH |
|---|---|---|---|---|---|
| α_{2B}(h) (antagonist effect) | 2.3E−04M | | 5.0E−05M | | |
| α_{2B}(h) (antagonist radioligand) | 3.0E−06M | 3.0E−06M | | | 0.7 |
| δ₂ (DOP) (antagonist effect) | 1.5E−05M | | 2.4E−06M | | |
| δ₂ (DOP) (h) (agonist | 4.1E−05M | 5.8E−05M | | | 1 |
| radioligand) | | | | | |
| κ (KOP) (agonist effect) | | | | 5.6E−04M | |
| κ (KOP) (agonist radioligand) | 8.4E−06M | 1.2E−05M | | | 1.1 |
| μ (MOP) (h) (agonist | 4.8E−07M | 1.1E−07M | | | 0.8 |
| radioligand) | | | | | |
| μ (MOP) (h) (antagonist effect) | 3.1E−05M | | 1.5E−06M | | |

### 8-bromo-N-(1-phenethylpiperidin-4-yl)octanamide

| Assay | IC₅₀ | Kᵢ | K_{B} | EC₅₀ | nH |
|---|---|---|---|---|---|
| α_{2B}(h) (antagonist effect) | 3.6E−04M | | 4.6E−05M | | |
| α_{2B}(h) (antagonist radioligand) | 5.7E−06M | 3.8E−06M | | | 0.8 |
| κ (KOP) (agonist radioligand) | 6.0E−05M | 4.0E−05M | | | 0.5 |
| μ (MOP) (h) (agonist radioligand) | | | | 8.0E−04M | |
| μ (MOP) (h) (antagonist effect) | 9.5E−05M | 3.9E−05M | | | 0.4 |

### N-(1-phenethylpiperidin-4-yl)benzamide

| Assay | IC₅₀ | Kᵢ | K_{B} | EC₅₀ | nH |
|---|---|---|---|---|---|
| α_{2B}(h) (antagonist effect) | 3.5E−04M | | 4.6E−05M | | |
| α_{2B}(h) (antagonist radioligand) | 4.0E−06M | .27E−06M | | | 1.3 |
| δ₂ (DOP) (antagonist effect) | 9.6E−05M | | 1.7E−06M | | |
| δ₂ (DOP) (h) (agonist | 8.7E−05M | 5.1E−05M | | | 0.7 |
| radioligand) | | | | | |
| κ (KOP) (agonist effect) | | | | 4.6E−04M | |
| κ (KOP) (agonist radioligand) | 3.2E−05M | 2.1E−05M | | | 0.6 |
| μ (MOP) (h) (agonist | 2.5E−06M | 1.0E−06M | | | 0.9 |
| radioligand) | | | | | |
| μ (MOP) (h) (antagonist effect) | 3.8E−05M | | 4.1E−06M | | |

### N-(1-(2-(thiophen-2-yl)ethyl) piperidin-4-yl)propionamide

| Assay | IC₅₀ | Kᵢ | K_{B} | EC₅₀ | nH |
|---|---|---|---|---|---|
| α_{2B}(h) (antagonist effect) | 4.4E−04M | | 5.7E−05M | | |
| α_{2B}(h) (antagonist radioligand) | 1.1E−05M | 7.6E−06M | | | 1.2 |
| δ₂ (DOP) (h) (agonist | 4.1E−04M | 2.5E−04M | | | 0.9 |
| radioligand) | | | | | |
| κ (KOP) (agonist effect) | | | | 3.1E−04M | |
| κ (KOP) (agonist radioligand) | 1.6E−05M | 1.1E−05M | | | 0.6 |
| μ (MOP) (h) (agonist | | | | 6.6E−06M | |
| radioligand) | | | | | |
| μ (MOP) (h) (antagonist effect) | 9.0E−07M | 3.7E−07M | | | 0.6 |

Results showing an inhibition (or stimulation for assays run in basal conditions) higher than 50% are considered to represent significant effects of the test compounds. Results showing an inhibition (or stimulation) between 25% and 50% are indicative of weak to moderate effects. Results showing an inhibition (or stimulation) lower than 25% are not considered significant. High negative values (≥50%) that are sometimes obtained with high concentrations of test compounds are generally attributable to nonspecific effects of the test compounds in the assays. On rare occasion they could suggest an allosteric effect of the test compound.

Mice in vivo Test: 16 female and 16 male CD-1 mice were baselined for 15 minutes in the conditioned place preference (CPP) chambers on day one. The mice then underwent four days of twice daily 30 minute drug conditioning sessions, receiving either Morphine (n=16) (10 mg/kg: SC), or Compound 1 (n=16) (10 mg/kg: IP) followed 10 minutes later by Morphine (10 mg/kg: SC). These drugs were paired with either the stripe or dot chambers. For the unpaired chambers the animals would receive vehicle (10% DMSO, 10% Tween80, 80% saline). After conditioning, the animals were free to roam the CPP chambers as they had during the baseline session for 15 minutes. Data was collected for the amount of time the animal spent in each chamber, excluding transition time and time spent in the corridor. One female animal in the Morphine group and one female animal in the Compound 1+Morphine group were excluded due to insufficient baseline measurements. One male animal was excluded from the experiment due to an infection unrelated to the experiment.

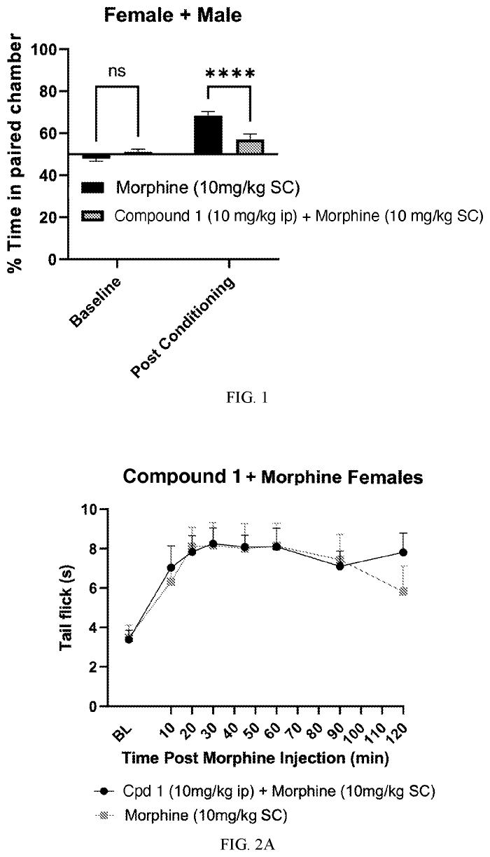

As shown in FIG. 1, the mice treated with a combination of compound 1 with morphine spent a significantly less time in a chamber associated with morphine administration indicating a significantly reduced addictive property of a composition of the invention.

Mouse Tail Flick Test: 10 CD-1 female and 10 CD-1 male mice were baselined on a Tail Flick assay to measure their response time to painful thermal stimuli. The animals tails were placed in water 52° C. Cut off time for the animals to have their tails submerged was 10 seconds to prevent tissue damage. The mice were then given compound 1 (10 mg/kg: IP) followed 10 minutes later by Morphine (10 mg/kg SC) n=10. Control animals were given only Morphine (10 mg/kg SC) n=10.

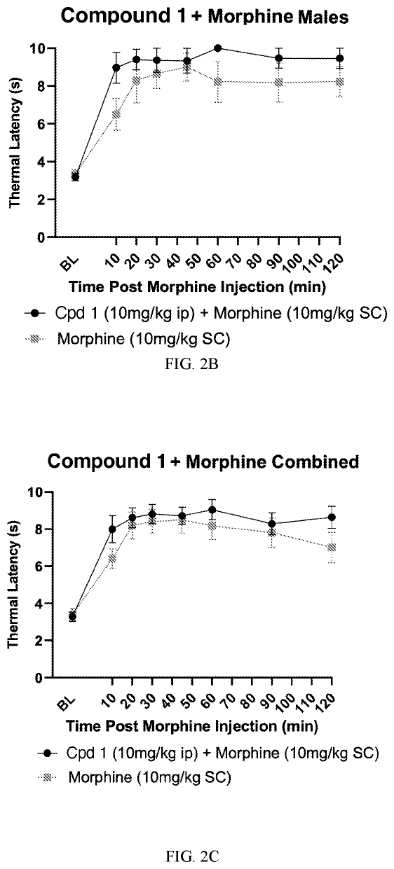

As shown in FIGS. 2A-2C, composition of the invention showed almost identical analgesic property compared to morphine alone.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A compound encompassed within: 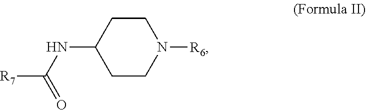
including pharmaceutically acceptable salts, solvates, and/or prodrugs thereof,
wherein each of R6 and R7 independently include any chemical moiety that permits the resulting compound to inhibit and/or antagonize adrenoeceptor activity

2. The compound of claim 1, wherein each of R6 and R7 independently include any chemical moiety that permits the resulting compound capable of one or more of the following:
inhibiting opioid receptor activity;
inhibiting α2A adrenoreceptor activity;
preventing and/or attenuating the addictive properties of opioid receptor agonist compounds;
treating, preventing or attenuating pain;
treating, preventing or attenuating nociceptive pain;
treating, preventing or attenuating neuropathic pain;
providing non-addictive pain relief to a subject upon co-administration with an opioid receptor agonist compound; and
providing direct treatment for opioid addiction disorder to a subject upon co-administration with an opioid receptor agonist compound.

3. The compound of claim 1, wherein R6 is selected from

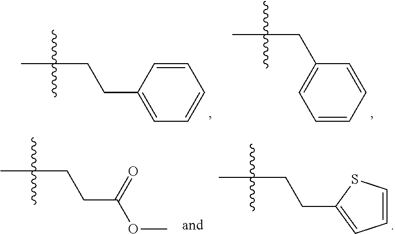

4. The compound of claim 1, wherein 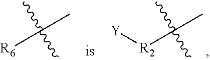
R2 is C₁₋₆ alkylene; Y is optionally substituted aryl, optionally substituted heteroaryl, or a moiety of the formula —C(═O)—X¹; X¹ is —OR³ or —NR⁴R⁵; and each of R³, R⁴ and R⁵ is independently H or C₁₋₁₀ alkyl.

5. The compound of claim 4, wherein R2 is ethylene.

6. The compound of claim 4, wherein Y is selected from phenyl, thiophen-2-yl, and a moiety of the formula —C(═O)—OR³, where R³ is C₁₋₁₀ alkyl.

7. The compound of claim 1, wherein R7 is selected from

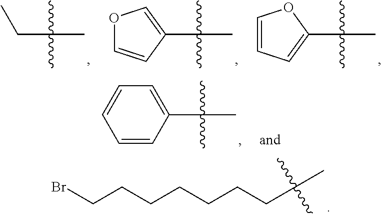

8. The compound of claim 1, wherein R7 is selected from C₁₋₁₀ alkyl, C₁₋₁₀ haloalkyl, optionally substituted aryl, and optionally substituted heteroaryl.

9. The compound of claim 1, R7 is selected from ethyl, 7-bromoheptyl, fur-2-yl, fur-3-yl, and phenyl.

10. The compound of claim 1, wherein the compound is selected from:

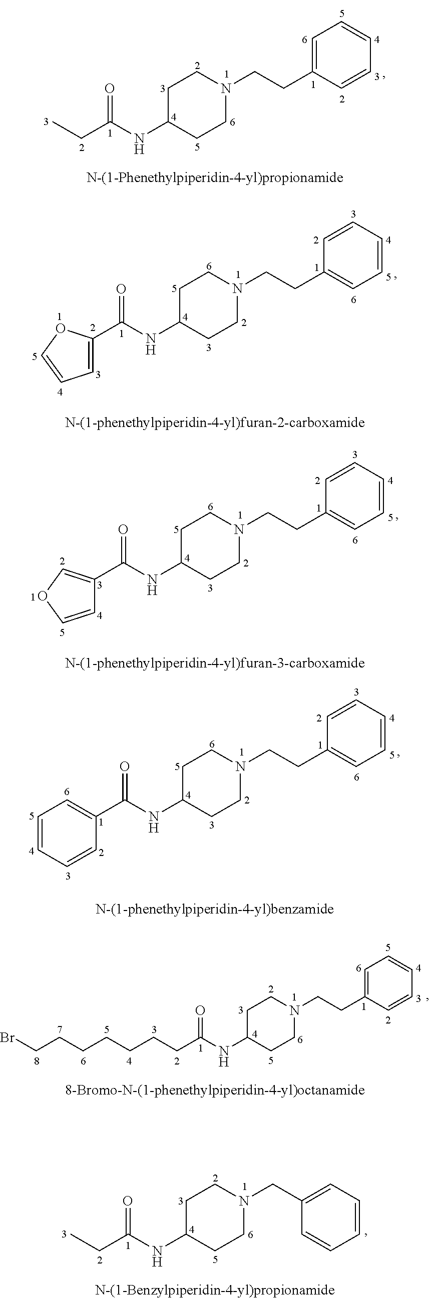 [SMILES: CCC(=O)NC1CCN(CCc2ccccc2)CC1 ; O=C(NC1CCN(CCc2ccccc2)CC1)c1ccco1 ; O=C(NC1CCN(CCc2ccccc2)CC1)c1ccoc1 ; O=C(NC1CCN(CCc2ccccc2)CC1)c1ccccc1 ; O=C(CCCCCCCBr)NC1CCN(CCc2ccccc2)CC1 ; CCC(=O)NC1CCN(Cc2ccccc2)CC1]

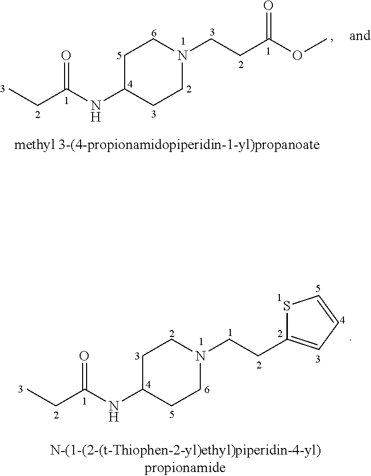

11. A pharmaceutical composition comprising a compound recited in claim 1.

12. A method of treating, ameliorating, or preventing a condition related to adrenoreceptor activity in a patient comprising administering to said patient a therapeutically effective amount of the pharmaceutical composition of claim 11.

13. The method of claim 12, wherein said condition related to adrenoreceptor activity is one or more of nociceptive pain and neuropathic pain.

14. The method of claim 12, wherein said patient is a human patient.

15. The method of claim 12, further comprising administering to said patient one or more agents for treating pain.

16. The method of claim 15, wherein the one or more agents for treating pain is one or more opioid receptor agonist compounds (e.g., morphine, fentanyl, carfentanyl, pentazocine, butorphanol, nalbuphine, buprenorphine, sufentanil, alfentanil, tramadol, remifentanil, hydrocodone, oxycodone, hydromorphone, oxymorphone, or similar clinically used opioid receptor agonist drug).

17. A method of treating, ameliorating, or preventing a condition related to adrenoreceptor activity in a patient comprising administering to said patient a therapeutically effective amount of a composition comprising
one or more compounds of claim 1, and
one or more opioid receptor agonist compounds (e.g., morphine, fentanyl, carfentanyl, pentazocine, butorphanol, nalbuphine, buprenorphine, sufentanil, alfentanil, tramadol, remifentanil, hydrocodone, oxycodone, hydromorphone, oxymorphone, or similar clinically used opioid receptor agonist drug).

18. The method of claim 17, wherein said condition related to adrenoreceptor activity is one or more of nociceptive pain and neuropathic pain.

19. The method of claim 17, wherein said patient is a human patient.

20. A composition comprising an opioid receptor agonist compound and an adrenoreceptor modulator compound.

21. The composition of claim 20, wherein said opioid receptor agonist compound comprises morphine, fentanyl, carfentanyl, pentazocine butorphanol, nalbuphine, buprenorphine, sufentanil, alfentanil, tramadol, remifentanil, hydrocodone, oxycodone, hydromorphone, or oxymorphone.

22. The composition of claim 20, wherein said adrenoreceptor modulator compound is an α-2A adrenoceptor modulator compound.

23. The composition of claim 20, wherein said adrenoreceptor modulator compound is of the formula: 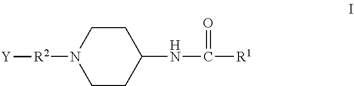
wherein
R¹ is C₁₋₁₀ alkyl, C₁₋₁₀ haloalkyl, optionally substituted aryl, or optionally substituted heteroaryl;
R² is C₁₋₆ alkylene; and
Y is optionally substituted aryl, optionally substituted heteroaryl, or a moiety of the formula —C(═O)—X¹, wherein X¹ is —OR³ or —NR⁴R⁵, where each of R³, R⁴ and R⁵ is H or C₁₋₁₀ alkyl.

24. The composition of claim 22,
wherein R¹ is selected from the group consisting of ethyl, 7-bromoheptyl, fur-2-yl, fur-3-yl, and phenyl,
wherein R² is ethylene.

25. The composition of claim 22, wherein Y is selected from the group consisting of phenyl, thiophen-2-yl, and a moiety of the formula —C(—O)—OR³, where R³ is C₁₋₁₀ alkyl.

26. The composition of claim 20, wherein said adrenoreceptor modulator compound comprises N-(1-phenethylpiperidin-4-yl)propionamide, N-(1-phenethylpiperidin-4-yl)furan-2-carboxamide, N-(1-phenethylpiperidin-4-yl)furan-3-carboxamide, or N-(1-(2-(thiophen-2-yl)ethyl) piperidin-4-yl)propionamide.

27. A method for treating pain or addiction disorder in a subject, said method comprising administering to a subject in need of such a treatment a therapeutically effective amount of a composition comprising an opioid receptor agonist compound and an adrenoreceptor modulator compound.

28. The method of claim 27, wherein said opioid receptor agonist compound comprises morphine, fentanyl, carfentanyl, pentazocine butorphanol, nalbuphine, buprenorphine, sufentanil, alfentanil, tramadol, remifentanil, hydrocodone, oxycodone, hydromorphone, or oxymorphone.

29. The method of claim 27, wherein said adrenoreceptor modulator compound is an α-2A adrenoceptor modulator compound.

30. The method of claim 27, wherein said adrenoreceptor modulator compound is of the formula: 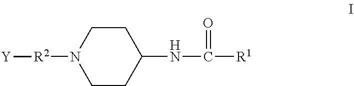
wherein
R¹ is C₁₋₁₀ alkyl, C₁₋₁₀ haloalkyl, optionally substituted aryl, or optionally substituted heteroaryl;
R² is C₁₋₆ alkylene; and
Y is optionally substituted aryl, optionally substituted heteroaryl, or a moiety of the formula —C(═O)—X¹, wherein X¹ is —OR³ or —NR⁴R⁵, where each of R³, R⁴ and R⁵ is H or C₁₋₁₀ alkyl.

31. The method of claim 30, wherein R¹ is selected from the group consisting of ethyl, 7-bromoheptyl, fur-2-yl, fur-3-yl, and phenyl.

32. The method of claim 30, wherein R² is ethylene.

33. The method of claim 30, wherein Y is selected from the group consisting of phenyl, thiophen-2-yl, and a moiety of the formula —C(═O)—OR³, where R³ is C₁₋₁₀ alkyl.

34. The method of claim 27, wherein said adrenoreceptor modulator compound comprises N-(1-phenethylpiperidin-4-yl)propionamide, N-(1-phenethylpiperidin-4-yl)furan-2-carboxamide, N-(1-phenethylpiperidin-4-yl)furan-3-carboxamide, or N-(1-(2-(thiophen-2-yl)ethyl) piperidin-4-yl)propionamide.

35. The method of claim 27, wherein said pain is chronic pain.

36. The method of claim 27, wherein said pain is acute pain.

37. The method of claim 27, wherein said pain is nociceptive pain.

38. The method of claim 27, wherein said pain is neuropathic pain.

39. A composition comprising an opioid receptor agonist compound and a second biologically active compound, wherein said second biologically active compound has an adrenoreceptor modulator activity and an opioid receptor modulating activity.

40. The composition of claim 39, wherein said second biologically active compound is of the formula: 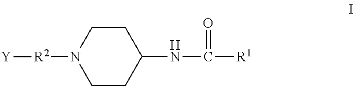
wherein
R¹ is C₁₋₁₀ alkyl, C₁₋₁₀ haloalkyl, optionally substituted aryl, or optionally substituted heteroaryl;
R² is C₁₋₆ alkylene; and
Y is optionally substituted aryl, optionally substituted heteroaryl, or a moiety of the formula —C(═O)—X¹, wherein X¹ is —OR³ or —NR⁴R⁵, where each of R³, R⁴ and R⁵ is H or C₁₋₁₀ alkyl.

41. The composition of claim 40, wherein R¹ is selected from the group consisting of ethyl, 7-bromoheptyl, fur-2-yl, fur-3-yl, and phenyl.

42. The composition of claim 40, wherein R² is ethylene.

43. The composition of claim 40, wherein Y is selected from the group consisting of phenyl, thiophen-2-yl, and a moiety of the formula —C(═O)—OR³, where R³ is C₁₋₁₀ alkyl.

44. The composition of claim 39, wherein said second biologically active compound comprises N-(1-phenethylpiperidin-4-yl)propionamide, N-(1-phenethylpiperidin-4-yl)furan-2-carboxamide, N-(1-phenethylpiperidin-4-yl)furan-3-carboxamide, or N-(1-(2-(thiophen-2-yl)ethyl)piperidin-4-yl)propionamide.
